(19) United States
(12) Patent Application Publication
(10) Pub. No.: US 2023/0212638 A1
(43) Pub. Date: Jul. 6, 2023

(54) MICROORGANISM TEST METHOD AND MICROORGANISM TEST APPARATUS

(71) Applicants: HYOGO COLLEGE OF MEDICINE, Hyogo (JP); KYOTO UNIVERSITY, Kyoto (JP)

(72) Inventors: Shojiro KIKUCHI, Nishinomiya-shi, Hyogo (JP); Yuichi OGAWA, Kyoto-shi, Kyoto (JP)

(21) Appl. No.: 17/927,984

(22) PCT Filed: May 28, 2021
(86) PCT No.: PCT/JP2021/020346

§ 371 Date: Mar. 7, 2023

(30) Foreign Application Priority Data
May 29, 2020 (JP) 2020-094081

(51) Int. Cl. C12Q 1/02; G01N 22/00; G01N 29/036

(52) U.S. Cl. CPC C12Q 1/02; G01N 22/00; G01N 29/036; G01N 2291/014

(57) ABSTRACT

A microorganism test method includes: covering, with a hydrophobic capping solvent, a sample containing a specimen and a liquid culture medium, within a region in a vicinity of a sensor configured to detect a microorganism contained in the specimen; and calculating, based on an output from the sensor, information indicating a degree of growth of the microorganism contained in the specimen. For example, an analysis unit drives an array sensor in which many resonators are arranged in a matrix pattern, stores a resonance frequency of a resonator which is acquired at the time of starting the measurement as an initial frequency, and calculates a difference (frequency shift) between the initial frequency and a resonance frequency of the resonator which is measured at predetermined time intervals as information indicating a degree of growth of the microorganism contained in the specimen.

### TECHNICAL FIELD

The present invention relates to a microorganism test method and a microorganism test apparatus, which are for testing a state of microorganisms in a specimen.

### BACKGROUND ART

At present, in the fields of medicine and agriculture, testing of microorganisms, which is rapid, efficient, and highly precise, is required. For example, for Mycobacterium tuberculosis that is a difficult-to-culture bacterium, it takes around two months to test the drug susceptibility using an agar medium or a liquid culture medium. For this reason, optimal and accurate drug administration is difficult, and as a result, various medications are repeated over a long period of time.

The following PTL 1 discloses a test method that uses terahertz waves to test the amount of the moisture contained in a cell. In this test method, the amount of the moisture in a cell is detected by frequency shift of terahertz waves, and based on the detection results, for example, canceration or the like of the cell is evaluated.

Further, the following PTL 2 discloses a sensor circuit including a cross-coupled oscillator that oscillates at a frequency of 30 to 200 GHz, and a detection unit that detects a change in the property of an inspection target referring to a change in the frequency of the oscillator. In this sensor circuit, a change in the inspection target containing moisture is detected by replacing the change in the inspection target with a change of bulk water in the moisture. That is, the decrease of bulk water by replacing water molecules with a protein is referred as a change in the frequency of an oscillator, and a change in the property of the inspection target is detected based on the change in the frequency.

### CITATION LIST

### Patent Literature

PTL 1: Japanese Patent Laying-Open No. 2019-60609

PTL 2: U.S. Pat. No. 6,416,398

### SUMMARY OF INVENTION

### Technical Problem

Microorganisms such as Mycobacterium tuberculosis and Escherichia coli are remarkably smaller than cells of human and animals, and thus, the amount of moisture in these microorganisms is also remarkably less than that of the cells. For this reason, in a case where the test method of PTL 1 is used for testing these microorganisms, frequency shift of terahertz waves becomes extremely small.

Further, PTL 2 discloses that a change in the property of the inspection target containing moisture is detected. However, PTL 2 does not particularly disclose about subjecting microorganisms in moisture to testing, and a method therefor.

Further, in testing of microorganisms, convection may occur in a sample due to temperature change or the like. In this case, microorganisms in a sample do not stay in the vicinity of a sensor, but move with the convection. For this reason, it is difficult to accurately test the state of microorganisms in a sample based on an output from the sensor.

In view of such a problem, an object of the present invention is to provide a microorganism test method and a microorganism test apparatus, which can accurately conduct testing of microorganisms.

### Solution to Problem

A microorganism test method according to the first aspect of the present invention includes: covering, with a hydrophobic capping solvent, a sample containing a specimen and a liquid culture medium, within a region in a vicinity of a sensor, the sensor configured to detect a microorganism contained in the specimen; and calculating, based on an output from the sensor, information indicating a degree of growth of the microorganism contained in the specimen.

According to the microorganism test method according to the present aspect, by reducing the influence of convection or the like due to temperature change, the sample can be kept in the region in the vicinity of the sensor. Accordingly, testing of a degree of growth of microorganisms in a sample can be more accurately conducted.

In this case, enclosing of a sample with a capping solvent may be conducted by introducing the sample in a storing region including the region in the vicinity of a sensor, and then by adding the capping solvent in the storing region, alternatively, may be conducted by introducing the capping solvent in a storing region including the region in the vicinity of a sensor, and then by adding the sample in the storing region.

In the latter case, in the microorganism test method, adjustment may be performed so that a sample is enclosed in the region in the vicinity of the sensor, for example, by introducing the capping solvent in the storing region including the region in the vicinity of the sensor, inserting an insertion tool into the capping solvent, and introducing the sample through the insertion tool in the region in the vicinity of the sensor.

In the microorganism test method according to the present aspect, the sensor can be an array sensor in which a plurality of sensor elements are arranged adjacently in a matrix pattern. As a result, the degree of growth of microorganisms can be tested for each position of the elements. Further, distribution of the degree of growth of microorganisms in the entire detection range of the array sensor can also be tested.

In this case, each of the sensor elements includes an oscillator that oscillates in a gigahertz band, and can be adjusted so that the shift of the oscillation frequency of the oscillator is calculated as the information indicating the degree of growth of the microorganisms contained in the specimen. In this way, the degree of growth of microorganisms can be accurately grasped by the shift of the oscillation frequency of the oscillator.

The microorganism test method according to the present aspect can include a step of displaying a growth curve for evaluating shift with time in the growth of microorganisms based on an output from the sensor. In this way, the tester can intuitively grasp the degree of growth of microorganisms by referring to the displayed growth curve.

In the microorganism test method according to the present aspect, the capping solvent has gas solubility, and can be adjusted so that the sample is enclosed in the region in the vicinity of the sensor with the capping solvent in which oxygen is dissolved. In this way, in a case where the microorganism is an aerobic microorganism such as Mycobacterium tuberculosis, oxygen can be supplied from a capping solvent to the microorganism in a sample enclosed with the capping solvent.

In this regard, as the capping solvent, a fluorine-based inert solvent or mineral oil can be used.

Further, the microorganism to be tested may include Mycobacterium tuberculosis. Mycobacterium tuberculosis does not have motility, but can easily move out of a region in the vicinity of a resonator due to the convection of a liquid culture medium. Accordingly, it is preferable that in order to prevent the convection of a liquid culture medium in testing of Mycobacterium tuberculosis, a sample is enclosed in a region in the vicinity of a sensor with a capping solvent.

In the microorganism test method according to the second aspect of the present invention, a sample prepared by mixing a specimen with a liquid culture medium is applied to a resonator that oscillates in a gigahertz band, and shift of a resonance frequency of the resonator is calculated as information indicating a degree of growth of microorganisms contained in the specimen.

A sample prepared by mixing a specimen with a liquid culture medium contains bulk water that is not bound to microbial biomolecules and bound water that is bound to microbial biomolecules. In this regard, respective dielectric losses of the bulk water and the bound water are different from each other. In addition, as the growth of microorganisms proceeds in a sample, biomolecules to which water molecules bind increase. For this reason, with the growth of microorganisms, the bulk water surrounding the microorganisms is bound to biomolecules and becomes bound water, and as a result, the ratio of the bulk water to the water surrounding the microorganisms is reduced. Accordingly, when microorganisms are proliferated, the dielectric loss of the water surrounding the microorganisms changes. In this regard, the dielectric loss of bulk water occurs in a gigahertz frequency band. Therefore, as described above, by applying a sample to a resonator that oscillates in a gigahertz band, a resonance frequency of the resonator shifts with the growth of microorganisms. By calculating this shift, the degree of growth of microorganisms in a sample can be grasped.

Therefore, according to the microorganism test method according to the present aspect, by applying a sample to a resonator that oscillates in a gigahertz band and calculating the shift of the resonance frequency, the degree of growth of microorganisms in the sample can be tested. Further, because there is a sufficient amount of water (bulk water, and bound water) in the surroundings of microorganisms, the shift of the resonance frequency of a resonator can be accurately matched with the degree of growth of microorganisms. Furthermore, because the degree of growth of microorganisms is quantified by the shift of the resonance frequency of a resonator, the degree of growth of microorganisms in a sample can be determined rapidly. Therefore, the testing of microorganisms can be accurately and rapidly conducted.

In this regard, the above gigahertz band is preferably greater than or equal to 10 GHz and less than or equal to 600 GHz. By setting the resonance frequency of a resonator to greater than or equal to 10 GHz, the influence of ions existing in a sample on the shift of the resonance frequency can be suppressed. In addition, by setting the resonance frequency of a resonator to less than or equal to 600 GHz, the decreased range of bulk water due to the growth of microorganisms can be made larger, and the shift range of the resonance frequency with the growth of microorganisms can be made larger.

Further, the above gigahertz band is more preferably greater than or equal to 30 GHz and less than or equal to 300 GHz. By setting the resonance frequency of a resonator to greater than or equal to 30 GHz, the influence of ions existing in a sample can be almost suppressed. In addition, by setting the resonance frequency of a resonator to less than or equal to 300 GHz, the resonator is easy to oscillate accurately at a predetermined resonance frequency.

In the microorganism test method according to the present aspect, adjustment may be performed so that the sample is applied to an array sensor in which elements that include the resonators are arranged adjacently in a matrix pattern. As a result, the degree of growth of microorganisms can be tested for each position of the elements. Further, distribution of the shift of the resonance frequency in the entire detection range of the array sensor can also be tested.

In the microorganism test method according to the present aspect, adjustment may be performed so that the sample is enclosed in a region in a vicinity of the resonator with a capping solvent that is hydrophobic. As a result, by reducing the influence of convection or the like due to temperature change, at least part of a sample can be kept in a region in the vicinity of the resonator. Accordingly, the shift of the resonance frequency corresponding to the degree of growth of microorganisms in a sample can be accurately calculated, and testing of microorganisms can be more accurately conducted.

In this regard, the capping solvent has gas solubility, and can be adjusted so that oxygen is dissolved in the capping solvent. As described above, in a case where the microorganism to be tested is an aerobic microorganism such as Mycobacterium tuberculosis, by dissolving oxygen in the capping solvent, oxygen can be supplied from the capping solvent to the sample enclosed with the capping solvent.

In this case, as the capping solvent, for example, a fluorine-based inert solvent or mineral oil can be used.

Further, the microorganism to be tested may include at least one of Mycobacterium tuberculosis, Escherichia coli, and Staphylococcus epidermidis. Mycobacterium tuberculosis does not have motility, but can easily move out of a region in the vicinity of a resonator due to the convection of a liquid culture medium. Therefore, in a case where the microorganism to be tested is Mycobacterium tuberculosis, it is preferable that the sample is enclosed in a region in the vicinity of the resonator by using the capping solvent. In contrast, because Escherichia coli has motility by itself and moves in the liquid culture medium, it can be distributed substantially evenly in the sample with the growth. Therefore, in a case where the microorganism to be tested is Escherichia coli, the degree of the growth can be tested by the shift of the resonance frequency without necessarily using a capping solvent. As described above, in the testing, it may be selected whether or not to add a capping solvent depending on the property of moving in the liquid culture medium of the microorganism to be tested.

Further, in the microorganism test method according to the present aspect, for the sample containing a drug for evaluating drug sensitivity to the microorganism, the resonance frequency of the resonator is calculated, and a growth curve indicating shift with time of the calculated resonance frequency may be displayed in a display.

Alternatively, in the microorganism test method according to the present aspect, a plurality of the samples that contain drugs at different concentrations from each other for evaluating the drug sensitivity to the microorganism are prepared, the shift of the resonance frequency of the resonator is calculated for each sample, and the calculated shift with time of the resonance frequency of each sample may be displayed in the display.

In this way, the tester can evaluate the drug sensitivity of a microorganism to the drug by referring to the displayed growth curve. For example, the tester can quantitatively evaluate the concentration-dependent action or time-dependent action of antimicrobial drugs with different action mechanisms by referring to the displayed growth curve. The term “concentration-dependent action” is referred to as an action in which the drug efficacy depends on the concentration of the drug, and the term “time-dependent action” is referred to as an action in which the drug efficacy is exerted after a predetermined period of time elapses.

The third aspect of the present invention relates to a microorganism test apparatus. The microorganism test apparatus according to this aspect includes a housing that stores a liquid, an introduction portion that introduces, into the housing, a sample prepared by mixing a specimen with a liquid culture medium, and a capping solvent that is hydrophobic, a sensor arranged close to the housing, and an analyzer that calculates, based on an output from the sensor, information indicating a degree of growth of microorganisms contained in the specimen. The analyzer causes the introduction portion to enclose the sample in a region in a vicinity of the sensor with the capping solvent, and calculates information indicating the degree of growth.

According to the microorganism test apparatus according to this aspect, in a similar manner to the microorganism test method according to the above first aspect, by enclosing a sample in the region in the vicinity of the sensor with the capping solvent, the influence of convection or the like due to temperature change is reduced, and the sample can be kept in the region in the vicinity of the sensor. Accordingly, testing of a degree of growth of microorganisms in a sample can be more accurately conducted.

The fourth aspect of the present invention relates to a microorganism test apparatus. The microorganism test apparatus according to this aspect includes a housing that stores a sample prepared by mixing a specimen with a liquid culture medium, a resonator that is arranged close to the housing and oscillates in a gigahertz band, and an analyzer that calculates, based on a resonance frequency of the resonator, information indicating a degree of growth of microorganisms contained in the specimen.

According to the microorganism test apparatus according to this aspect, in a similar manner to the microorganism test method according to the above second aspect, by applying the sample to the resonator that oscillates in a gigahertz band, information indicating the degree of growth of the microorganisms in the sample can be accurately and rapidly calculated.

In each aspect above, the term “region in the vicinity of a sensor” means a region where an object to be tested can be detected by the sensor, the term “capping solvent” means a solvent for capping a sample, and the term “capping” means enclosing a sample in a region in the vicinity of a sensor with a capping solvent.

### Advantageous Effects of Invention

As described above, according to the present invention, a microorganism test method and a microorganism test apparatus, which can accurately conduct a test of a microorganism can be provided.

The effects and significance of the present invention will become clearer from the description of embodiments shown below. However, the embodiments shown below are merely examples for carrying out the present invention, and the present invention should not be limited to the following embodiments.

### BRIEF DESCRIPTION OF DRAWINGS

FIG. 1 is a diagram showing the configuration of the microorganism test apparatus, according to Embodiment 1.

FIG. 2 is a diagram showing the configuration of the sensor circuit, according to Embodiment 1.

FIG. 3(a) is a plan view showing the configuration of the array sensor, according to Embodiment 1. FIG. 3(b) is a partial plan view showing an enlarged part of one element on the array sensor, according to Embodiment 1.

FIG. 4(a) is a perspective view schematically showing a state that a sample is applied to a region in the vicinity of one element of the array sensor, according to Embodiment 1. FIG. 4(b) is a diagram schematically showing changes of the resonance frequency of an element before and after the change of dielectric constant of a sample applied to the region in the vicinity, according to Embodiment 1.

FIGS. 5(a) and 5(b) are diagrams schematically showing molecular binding states of bulk water and bound water, respectively, according to Embodiment 1.

FIG. 6(a) is a graph showing the dielectric loss of bulk water, according to Embodiment 1. FIG. 6(b) is a graph showing the dielectric loss of biological water surrounding microorganisms, according to Embodiment 1.

FIGS. 7(a) to 7(d) are diagrams showing steps of adding a capping solvent to a sample, respectively, according to Embodiment 1. FIG. 7(e) is a sectional view schematically showing a state of a sample enclosed with a capping solvent, according to Embodiment 1.

FIG. 8 is a flowchart showing steps of the microorganism test method, according to Embodiment 1.

FIG. 9(a) is a diagram showing a configuration example of a display image in which the shift of the resonance frequency in each element is mapped to the position of each element, according to Embodiment 1. FIG. 9(b) is a graph showing a tendency of the calculation results when the shift of the resonance frequency is calculated in an element region where growth of microorganisms is observed and in an element region where growth of microorganisms is not observed, according to Embodiment 1.

FIGS. 10(a) to 10(c) are graphs showing the experimental results of the drug sensitivity of BCG, according to Example 1.

FIG. 11(a) is a graph showing the experimental results of the sensitivity of a concentration-dependent drug to NBRC3301 (Escherichia coli control), according to Example 2. FIGS. 11(b) and 11(c) are diagrams showing display images in each of which a frequency shift of each element measured in an experiment is displayed by color, respectively, according to Example 2.

FIG. 12(a) is a graph showing the experimental results of the sensitivity of a time-dependent drug to NBRC12993 (Staphylococcus epidermidis control), according to Example 3. FIGS. 12(b) and 12(c) are diagrams showing display images in each of which a frequency shift of each element measured in an experiment is displayed by color, respectively, according to Example 3.

FIG. 13(a) is a diagram schematically showing the configuration used in an experiment of dissolution of oxygen or nitrogen in a capping solvent, according to Example 4. FIG. 13(b) is a graph showing the experimental results of dissolution of oxygen or nitrogen in a capping solvent, according to Example 4.

FIGS. 14(a) to 14(d) are diagrams showing enclosing steps of enclosing a sample with a capping solvent, respectively, according to Embodiment 2. FIG. 14(e) is a sectional view schematically showing a state of a sample enclosed with a capping solvent, according to Embodiment 2.

FIG. 15 is a flowchart showing steps of the microorganism test method, according to Embodiment 2.

FIG. 16 is a diagram showing the configuration of the microorganism test apparatus, according to Embodiment 3.

FIGS. 17(a) to 17(d) are diagrams showing steps of adding a capping solvent to a sample, respectively, according to Embodiment 4.

FIG. 18(a) is a graph showing the experimental results of frequency shifts of the elements to be analyzed, according to Example 6. FIG. 18(b) is a display image showing the frequency shift of each element by color, according to Example 6.

FIG. 19(a) is a graph showing the experimental results of frequency shifts of the elements to be analyzed, according to Example 5. FIG. 19(b) is a display image showing the frequency shift of each element by color, according to Example 5.

FIG. 20(a) is a graph showing the experimental results of frequency shifts of the elements to be analyzed, according to Example 7. FIG. 20(b) is a display image showing the frequency shift of each element by color, according to Example 7.

FIGS. 21(a) to 21(c) are photomicrographs of measurement samples, respectively, according to Examples 6, 5, and 7.

In this regard, the drawings are for illustrative purposes only and do not limit the scope of the invention.

### DESCRIPTION OF EMBODIMENTS

### Embodiment 1

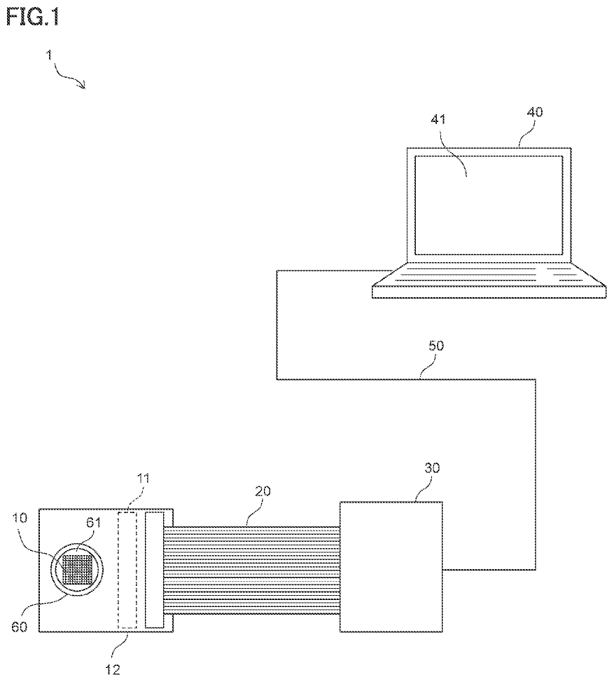

FIG. 1 is a diagram showing the configuration of a microorganism test apparatus 1, according to the present embodiment.

Microorganism test apparatus 1 includes an array sensor 10, a control unit 30, an analysis unit 40, and a container 60. In array sensor 10, elements that include resonators are arranged adjacently in a matrix pattern. Control unit 30 controls array sensor 10 in response to a command from analysis unit 40. Analysis unit 40 analyzes a degree of growth of microorganisms corresponding to a resonance frequency of each element input from control unit 30, and displays the analysis results on a monitor (display) 41. Analysis unit 40 is configured of, for example, a personal computer. In this case, an application program for analyzing a degree of growth of microorganisms is installed in the personal computer.

Array sensor 10 is configured of, for example, a complementary metal oxide semiconductor (CMOS), and mounted on a substrate 12. A circuit portion 11 including a driver and the like of array sensor 10 is further mounted on substrate 12. Circuit portion 11 and control unit 30 are connected by a signal cable 20 so as to be communicable with each other. Control unit 30 and analysis unit 40 are connected by a signal cable 50.

Container 60 is formed of a bottomless cylindrical member, and has a housing 61 for storing a liquid therein. Array sensor 10 is attached onto a bottom surface of container 60 in a state that the surface of array sensor 10 is sealed with a waterproof protective film. Array sensor 10 is water-tightly attached onto a bottom surface of container 60 with a protective film interposed therebetween so that a liquid stored in housing 61 does not leak. Container 60 may be a bottomed container. In this case, the thickness of the bottom part of container 60 is set thinly so that array sensor 10 can sense housing 61. In this configuration, array sensor 10 can be directly attached onto a bottom surface of container 60, and the protective film that seals a surface of array sensor 10 can be omitted. Housing 61 may have any size as long as it accommodates array sensor 10. Further, container 60 may have a configuration other than the above configuration as long as a liquid does not leak out when the liquid is stored in housing 61.

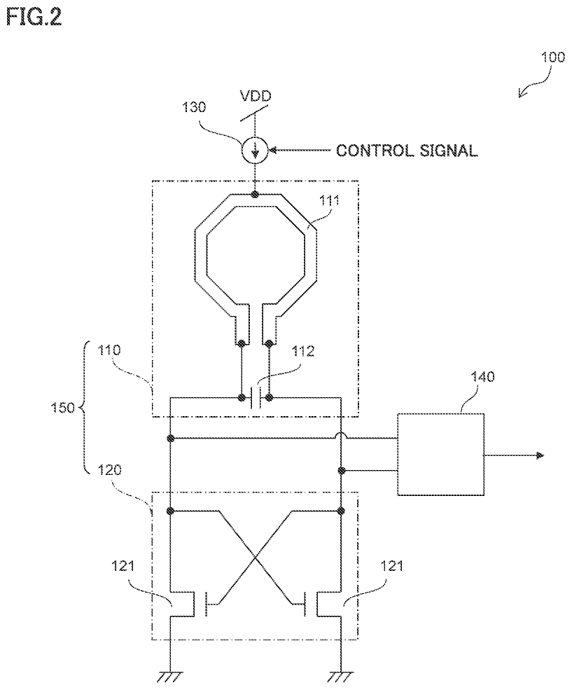

FIG. 2 is a diagram showing the configuration of a sensor circuit 100 of array sensor 10.

Sensor circuit 100 includes a resonator 110, a differential circuit 120, a current source 130, and a divider circuit 140.

Resonator 110 includes a loop-shaped inductor 111, and a capacitor 112. The inductance of inductor 111 and the capacitance of capacitor 112 determine the resonance frequency of resonator 110. Resonator 110 resonates at a predetermined resonance frequency included in a gigahertz band. In this regard, the configuration of resonator 110 is not limited to the configuration of FIG. 2. For example, the number of loops of inductor 111 does not have to be one, and inductor 111 may loop multiple times. Further, capacitor 112 may be configured of both ends of inductor 111 that are close to each other, or may be configured of parasitic capacitance of wiring (not shown) or the like.

Differential circuit 120 is configured by cross-coupling two transistors 121. The configuration of differential circuit 120 is not limited to this, and other configurations may be used. Current source 130 supplies an enable signal to an oscillator 150 including resonator 110 and differential circuit 120 in accordance with a control signal from circuit portion 11. In this way, resonator 110 and oscillator 150 oscillate at the above-predetermined resonance frequency included in a gigahertz band. When the resonance frequency of resonator 110 shifts, oscillator 150 oscillates at a resonance frequency after the shift and outputs a signal.

Divider circuit 140 divides the frequency of the resonance signal of resonator 110 to around one several hundredth, and outputs the frequency-divided signal. That is, divider circuit 140 divides the frequency of the resonance signal in a gigahertz band into resonance signals in a megahertz band and outputs the signals. The frequency-divided signal is supplied to analysis unit 40 via control unit 30 in FIG. 1. Analysis unit 40 calculates a resonance frequency of resonator 110 by multiplying the frequency of the input signal by a frequency division ratio.

In this regard, divider circuit 140 does not necessarily have to be arranged for each resonator 110. For example, in a case where the signal of each resonator 110 is individually processed by time division, the signal of each resonator 110 may be processed by one divider circuit 140 by time division. Further, divider circuit 140 may be arranged on the control unit 30 side.

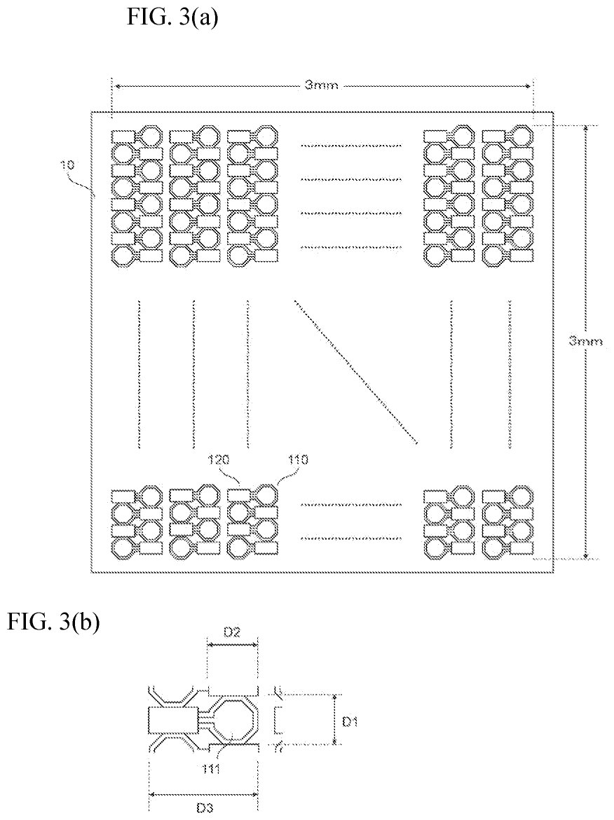

FIG. 3(a) is a plan view showing the configuration of array sensor 10. FIG. 3(b) is a partial plan view showing an enlarged part of one element on array sensor 10.

As shown in FIG. 3(a), in array sensor 10, elements that include resonators 110 of FIG. 2 are arranged adjacently in a matrix pattern. The size (diameter) of inductor 111 is set to a size with which resonance can be realized in a gigahertz band. For example, in a case where resonator 110 resonates at 64 GHz, the vertical and horizontal widths D1 and D2 of inductor 111 are set to around 45 μm and 51 μm, respectively in FIG. 3(b). In this case, the entire length D3 of oscillator 150 including resonator 110 and differential circuit 120 is around 110 μm.

In array sensor 10, for example, 1488 resonators 110 are arranged so as to be 62 rows and 24 columns. In this case, as shown in FIG. 3(a), the vertical and horizontal sizes of array sensor 10 are each around 3 mm. In this regard, the sizes of each portion shown in FIGS. 3(a) and 3(b) and the number of resonators 110 arranged in array sensor 10 are merely examples, and the present invention is not limited thereto.

By using array sensor 10 shown in FIG. 3(a), a sample can be applied to a large number of resonators 110 arranged in a matrix pattern.

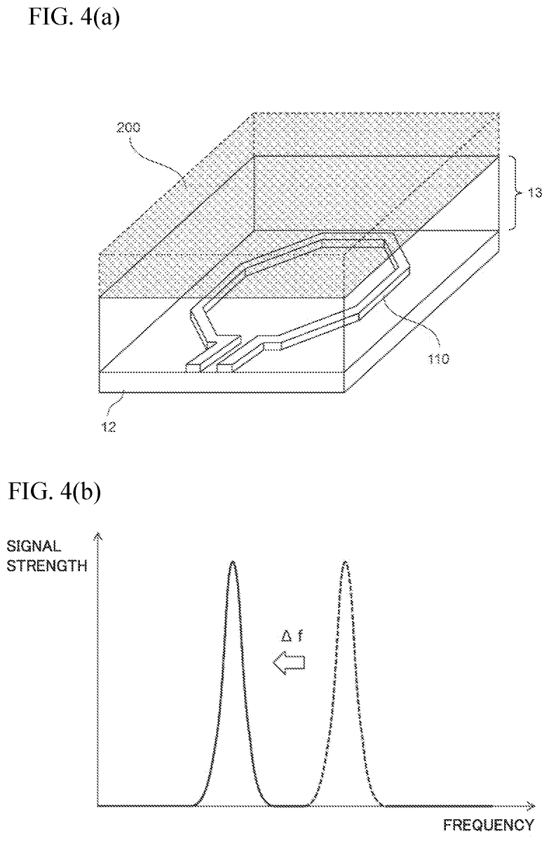

FIG. 4(a) is a perspective view schematically showing a state that a sample 200 is applied to a region in the vicinity of one element of the array sensor 10. FIG. 4(b) is a diagram schematically showing changes of the resonance frequency of an element before and after the change of dielectric constant of sample 200 applied to the region in the vicinity.

As shown in FIG. 4(a), the top surface of resonator 110 is covered with protective film 13 for waterproofing. Accordingly, the capacitance of protective film 13 is added as a parasitic capacitance for determining the resonance frequency of resonator 110. Further, in a case where sample 200 is applied onto protective film 13, the capacitance of sample 200 in a region close to (region in the vicinity of) resonator 110 is added as a parasitic capacitance for determining the resonance frequency of resonator 110. Therefore, in a case where sample 200 is applied, the resonance frequency of resonator 110 is a frequency determined by adding the capacitances of protective film 13 and sample 200.

In this regard, when the dielectric constant of sample 200 changes, the parasitic capacitance from sample 200 applied to resonator 110 (inductor 111) changes. For this reason, as shown in FIG. 4(b), along with the changes in the dielectric constant of sample 200, the resonance frequency of resonator 110 shifts only by Δf.

In the present embodiment, in sample 200, the growth of microorganisms is tested by utilizing the fact that the dielectric constant of the water surrounding microorganisms changes as the microorganism proliferates. Specifically, after applying sample 200 prepared by mixing a specimen with a liquid culture medium to array sensor 10, the shift of the resonance frequency (frequency shift Δf) in each element is acquired in time series, and the degree of growth of microorganisms is tested based on the acquired amount of shift. Hereinafter, the test method will be described.

First, the kind of the water existing in the surroundings of microorganisms and the dielectric loss of the water will be described.

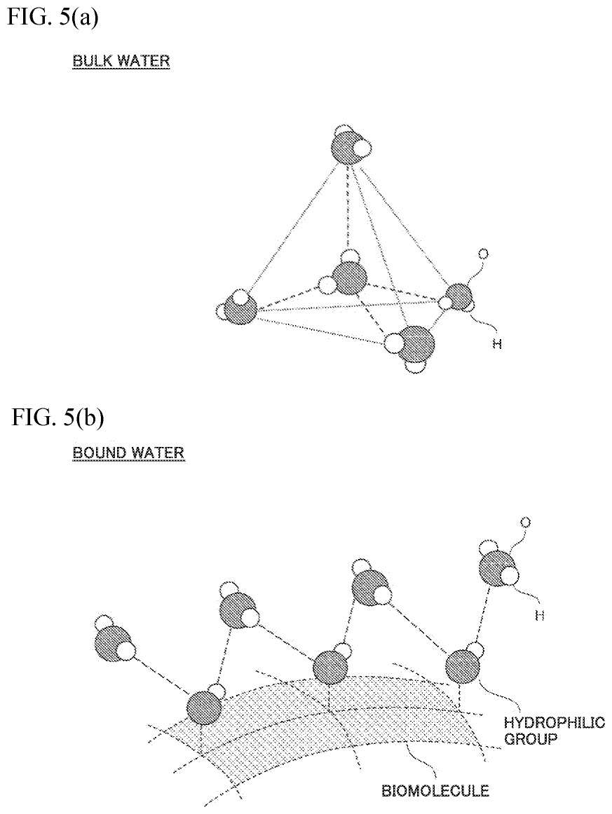

FIGS. 5(a) and 5(b) are diagrams schematically showing molecular binding states of bulk water and bound water, respectively.

Sample 200 prepared by mixing a specimen with a liquid culture medium contains bulk water, and bound water. Between them, as shown in FIG. 5(a), the bulk water is formed by tetrahedral intermolecular bonding of water molecules, and has a lifetime of around subpicoseconds to picoseconds. The bulk water moves freely in sample 200 because it is not bound to biomolecules. On the other hand, as shown in FIG. 5(b), the bound water is water in which water molecules are bound to biomolecules, and has a lifetime of around nanoseconds to microseconds.

The bulk water existing in the surroundings of microorganisms binds to microorganisms and becomes bound water, and after that, when the lifetime as the bound water ends, the bound water separates from the microorganisms, and converts to bulk water again. In this regard, the dielectric losses of the bulk water and the bound water are different from each other. Therefore, when the ratio of the bulk water to the water surrounding microorganisms changes, the dielectric constant of the water surrounding microorganisms changes, and in response to this, the resonance frequency of resonator 110 changes.

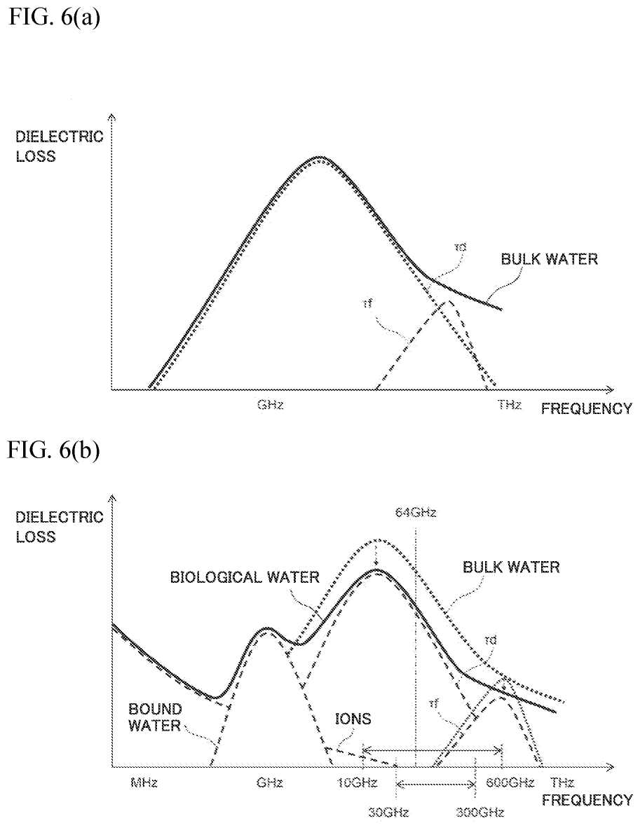

FIG. 6(a) is a graph showing the dielectric loss of bulk water. FIG. 6(b) is a graph showing the dielectric loss of biological water surrounding microorganisms. The horizontal axes in FIGS. 6(a) and 6(b) are each a logarithmic axis.

As shown in FIG. 6(a), the dielectric loss of bulk water occurs mainly in a gigahertz band ranging from 1 to 800 GHz. Therefore, by setting the resonance frequency of resonator 110 to a frequency included in a gigahertz band, changes of the dielectric constant of water based on changes of the dielectric loss of bulk water can be detected. In FIG. 6(a), the expression “τd” indicates the waveform of dielectric loss due to free water relaxation, and the expression “τf” indicates the waveform of dielectric loss due to intermolecular stretching vibration. In this regard, the dielectric loss due to other parameters exists also in a frequency band higher than the frequency band of the intermolecular stretching vibration, but this is omitted from the drawing.

As shown in FIG. 6(b), in the water (biological water) surrounding microorganisms, there is a frequency band of dielectric loss due to the above-described bound water, ions, or the like, in addition to bulk water. In this regard, as the growth of microorganisms proceeds, the biomolecules to which water molecules bind increase. For this reason, with the growth of microorganisms, the bulk water surrounding the microorganisms is bound to biomolecules and becomes bound water, and as a result, the ratio of the bulk water to the water surrounding the microorganisms is reduced. Therefore, when microorganisms are proliferated, as indicated by an arrow with a dashed line in FIG. 6(b), the dielectric loss of bulk water decreases and the dielectric loss of water surrounding the microorganisms changes.

In this regard, as shown in FIGS. 6(a) and 6 (b), the dielectric loss of bulk water occurs in a gigahertz frequency band. Therefore, as described above, by applying a sample to a resonator that oscillates in a gigahertz band, a resonance frequency of the resonator 110 shifts with the growth of microorganisms. By calculating this shift, the degree of growth of the microorganisms in sample 200 can be grasped.

Further, as shown in FIG. 6(b), the band range of the dielectric loss of bulk water partially overlaps with the band range of the dielectric loss of ions. For this reason, if the resonance frequency of resonator 110 is set in a frequency band with which the dielectric loss of ions overlaps, the resonance frequency of resonator 110 is affected by the ions contained in sample 200, and it becomes difficult to allow the shift of the resonance frequency to accurately correspond to the decrease in bulk water accompanying the growth of microorganisms. Therefore, in order to allow the growth of microorganisms to accurately correspond to the shift of the resonance frequency, it is preferable to set the resonance frequency of resonator 110 to a wavelength band of greater than or equal to 10 GHz, which is less affected by ions, and it is more preferable to set the resonance frequency of resonator 110 to a wavelength band of greater than or equal to 30 GHz, which is hardly affected by ions.

In addition, as shown in FIG. 6(b), even in the gigahertz frequency band, as the frequency comes closer to one terahertz, the range of the decrease in dielectric loss due to the decrease in bulk water is gradually reduced. For this reason, when the resonance frequency of resonator 110 comes closer to one terahertz, the decrease in bulk water with the growth of microorganisms is less likely to be effectively reflected on the shift of the resonance frequency. Therefore, in order to efficiently reflect the growth of microorganisms on the shift of the resonance frequency and to properly test the growth of microorganisms, it is preferable to set the resonance frequency of resonator 110 to less than or equal to 600 GHz.

Further, if the resonance frequency of resonator 110 exceeds 300 GHz, inductor 111 has to be remarkably miniaturized, and it becomes difficult to resonate resonator 110 at a predetermined resonance frequency also in terms of the circuit. For this reason, in order to properly resonate resonator 110 at a predetermined resonance frequency and to accurately test the growth of microorganisms, it is more preferable to set the resonance frequency of resonator 110 to less than or equal to 300 GHz.

For example, the resonance frequency of resonator 110 is set to 64 GHz. In this way, the decrease in bulk water with the growth of microorganisms can be accurately and efficiently reflected on the shift of the resonance frequency of resonator 110.

Next, a step of enclosing sample 200 in a region in the vicinity of array sensor 10 will be described.

In a case where resonator 110 resonates in a gigahertz band, the range of sample 200 that can affect the resonance frequency of resonator 110 is limited to a range of bottom part close to resonator 110. In other words, a region in the vicinity of above array sensor 10 is a detection region of a frequency shift with the growth of microorganisms. Therefore, it is preferable to keep sample 200 in this detection region during the test. For example, a microorganism which does not move around such as Mycobacterium tuberculosis may float in the upper part of sample 200 and move away from the region in the vicinity of array sensor 10 due to the convection or the like of sample 200 based on temperature change or the like. In such a situation, it becomes difficult to accurately test the growth of microorganisms.

In view of this, in the present embodiment, a capping solvent is added to sample 200 at the time of a test, and part of sample 200 is enclosed in a region in the vicinity of array sensor 10 with the capping solvent.

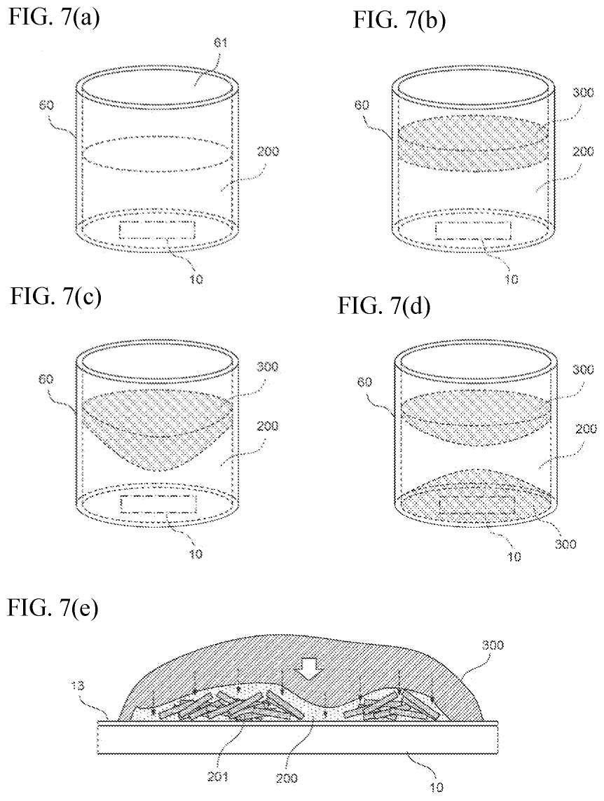

FIGS. 7(a) to 7(d) are diagrams showing steps of adding capping solvent 300 to sample 200, respectively. FIG. 7(e) is a sectional view schematically showing a state of sample 200 enclosed with capping solvent 300.

First, as shown in FIG. 7(a), sample 200 is introduced into container 60. Array sensor 10 is arranged on a bottom surface of container 60 in a state that the top surface of array sensor 10 is covered with protective film 13 (see FIG. 4(a)).

Next, as shown in FIG. 7(b), hydrophobic capping solvent 300 that has a specific gravity higher than that of water is added to sample 200. It is preferable that capping solvent 300 has low intermolecular force and low surface tension, and further has gas solubility. In a case where the microorganism to be tested is aerobic, oxygen is dissolved in capping solvent 300 in advance. As capping solvent 300, for example, a fluorine-based inert solvent can be used. For example, a perfluoro compound (PFC) can be used as capping solvent 300. In addition to this, silicone oil or the like can be used as capping solvent 300.

After that, as shown in FIG. 7(c), capping solvent 300 hangs down on the lower side of sample 200 due to own weight, and further, as shown in FIG. 7(d), part of capping solvent 300 separates and covers the bottom surface of container 60. As a result, as shown in FIG. 7(e), part of sample 200 is enclosed in a region in the vicinity of above array sensor 10 with capping solvent 300, and microorganisms 201 in sample 200 are retained in the region in the vicinity of array sensor 10. At this moment, by the pressure of capping solvent 300 due to own weight, microorganisms 201 together with sample 200 are pressed against the upper surface of array sensor 10 (protective film 13). Further, in a case where the microorganisms in sample 200 are aerobic, the gas (oxygen) dissolved in capping solvent 300 is eluted into sample 200, and the oxygen is supplied to microorganisms 201 in sample 200. In this way, the growth of microorganisms can be accurately tested by a frequency shift of each resonator 110 of array sensor 10.

In this regard, in a case where a microorganism which moves around such as Escherichia coli in sample 200 is an object to be tested, the addition of capping solvent 300 does not necessarily have to be performed. That is, in a case where the microorganism moves around in sample 200, with the growth of the microorganism, microorganisms can be distributed substantially evenly in a sample, and thus, the density of the microorganisms with the growth may change in a region in the vicinity of array sensor 10 even without using capping solvent 300. Therefore, in this case, the degree of growth can be tested by the shift of the resonance frequency without using capping solvent 300.

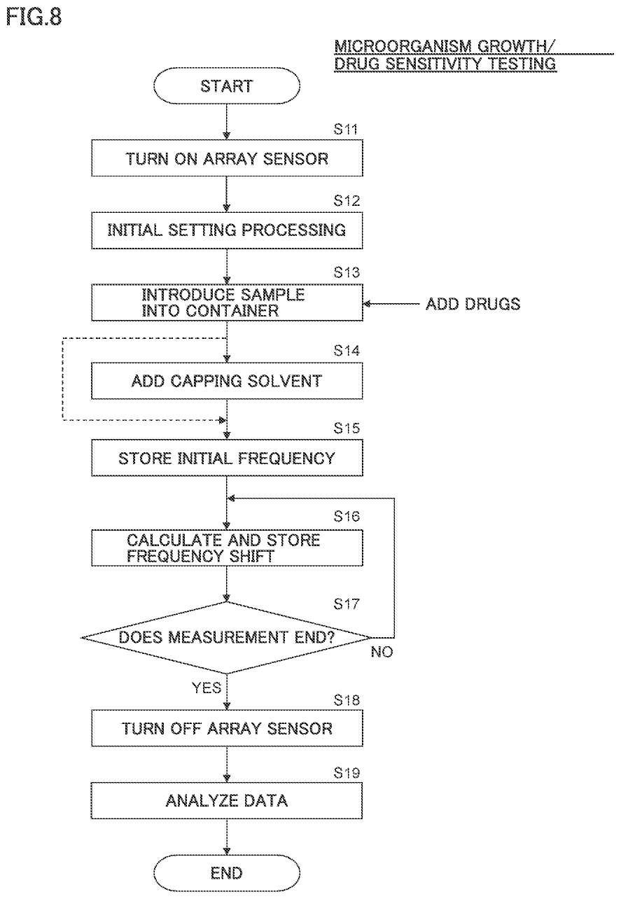

FIG. 8 is a flowchart showing steps of the microorganism test method.

First, the tester inputs the start of testing to analysis unit 40 in a state that container 60 shown in FIG. 7(a) is empty. Consequently, a command of the start of testing is output from analysis unit 40 to control unit 30, and array sensor 10 is activated (S11).

Next, analysis unit 40 executes initial setting processing of array sensor 10 (S12). In this processing, analysis unit 40 acquires characteristics of each element (resonator 110) on array sensor 10. That is, each element of array sensor 10 may contain inherent variations in the resonance frequency and the changing trend of the resonance frequency. Further, under the influence of ambient temperature, humidity, and the like, variations may occur in the resonance frequency and the changing trend of the resonance frequency, for each element. In order to correct such variations, analysis unit 40 acquires the resonance frequency and the changing trend of the resonance frequency, for each element, and a correction coefficient is set for each element so that the measurement operation of each element becomes uniform.

Specifically, analysis unit 40 measures the resonance frequency of each element the predetermined number of times while container 60 is empty, and further, the tester introduces ultrapure water into container 60, and the resonance frequency of each element is measured the predetermined number of times. In addition, analysis unit 40 compares for each element the resonance frequency when container 60 is empty with the resonance frequency when ultrapure water is introduced into container 60, and a correction coefficient when a frequency shift is calculated is set for each element.

After that, analysis unit 40 prompts the tester to introduce a sample into container 60 via a display screen or the like. In response to this, the tester discards the ultrapure water from container 60, and introduces into container 60 a sample prepared by mixing a pretreated specimen with a liquid culture medium suitable for the microorganism to be tested (S13).

In this way, when the sample is introduced into container 60, analysis unit 40 further prompts the tester to add capping solvent 300 into container 60. In response to this, the tester adds capping solvent 300 into container 60 (S14). Consequently, as shown in FIGS. 7(a) to 7(d), sample 200 is enclosed in a region in the vicinity of array sensor 10 with capping solvent 300. In addition, as described above, in a case where an object to be tested is a microorganism that does not require enclosing, a step of step S14 may be omitted as indicated with a dashed line in FIG. 8.

After that, analysis unit 40 executes measurement of a frequency shift on sample 200. Specifically, analysis unit 40 stores the resonance frequency of each element at the time of starting the measurement as the initial frequency (S15). Next, analysis unit 40 acquires the resonance frequency of each element at predetermined time intervals (for example, every minute), and calculates a difference between the acquired resonance frequency and the resonance frequency stored in step S15 for each element. Further, analysis unit 40 stores the calculated difference as the frequency shift in each element (S16). In this regard, the frequency shift is calculated, for example, by subtracting the resonance frequency acquired in step S16 from the initial frequency stored in step S15.

Analysis unit 40 repeats the processing of step S16 at predetermined time intervals until the measurement is completed (S17), and accumulates the frequency shift of each element. After that, when the input of the end of the measurement is received from the tester (S17: YES), analysis unit 40 stops array sensor 10 (S18), and executes analysis processing of the data accumulated in step S16 (S19). In this regard, the measurement may be completed when a predetermined set time (for example, several tens of hours) has elapsed from the start of measurement.

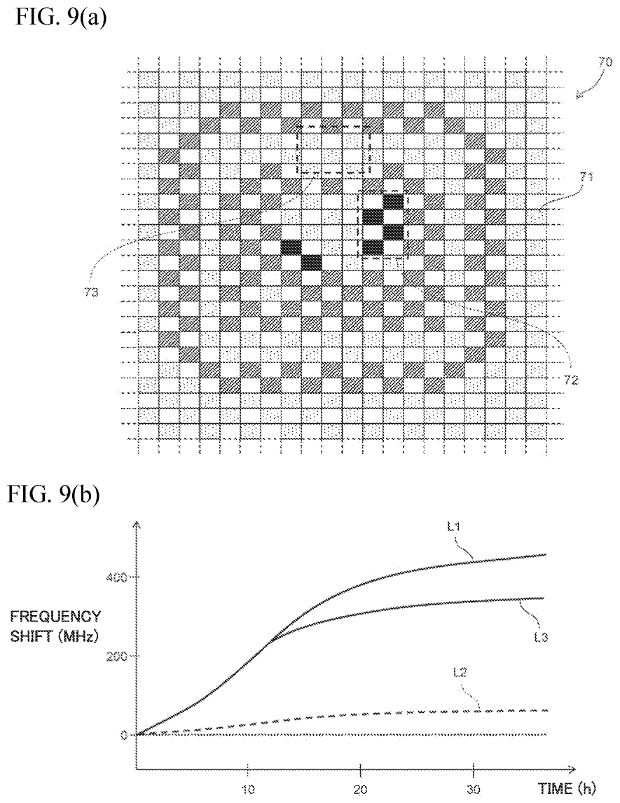

In the analysis processing in step S19, analysis unit 40 displays, for example, a display image 70 as shown in FIG. 9(a) on a monitor 41. Display image 70 is an image obtained by mapping a display color corresponding to the magnitude of the frequency shift calculated for each element in the position of each element. In display image 70, a cell 71 indicates a position of an element (inductor 111). For example, a display color is assigned to each cell 71 such that the display color changes from red through yellow to blue as the frequency shift moves from the maximum value to the minimum value. In FIG. 9(a), for convenience, the frequency shift is expressed by the density of hatching. In this case, the closer to black, the greater the frequency shift.

Analysis unit 40, for example, generates display image 70 and displays display image 70 on monitor 41, based on the frequency shift of each element acquired in the final step S16. With reference to display image 70, the tester can confirm the state of growth of microorganisms on array sensor 10 and the region where the growth of microorganisms has proceeded. In this regard, analysis unit 40 may be capable of changing the timing of generating display image 70 in response to an operation by the tester. In this way, the tester can confirm the state of growth of microorganisms at each timing.

Further, when the tester designates a predetermined cell region in display image 70, analysis unit 40 displays on monitor 41 a graph showing the changes with time in the frequency shift in the cell region. For example, when the tester designates a region 72 in display image 70 of FIG. 9(a), analysis unit 40 averages the frequency shifts of the elements corresponding to four cells included in region 72 for each timing of measurement, and displays on monitor 41 a graph in which an averaged frequency shift is plotted at each timing of the measurement.

In this way, for example, a graph (growth curve) as shown in FIG. 9(b) is displayed on monitor 41. L1 is, for example, a graph in a case where region 72 of FIG. 9(a) is designated. In addition, a graph in a case where a region 73 of FIG. 9(a) is designated, for example, a graph L2 of FIG. 9(b) is displayed on monitor 41. Further, in a case where a time-dependent drug is added into sample 200, changes with time of the frequency shift in a growth region of microorganisms are, for example, a graph L3 as shown in FIG. 9(b). With reference to such a graph, the tester can grasp the degree of growth of microorganisms in each region on array sensor 10.

In a case where the test method of FIG. 8 is used for a drug sensitivity testing of a microorganism, in step S13, the tester mixes a drug together with a specimen and a liquid culture medium to prepare sample 200, and introduces the prepared sample 200 into container 60. Other steps are similar to the above.

In a case where a microorganism is sensitive to the drug, the growth of the microorganism is suppressed in sample 200. Therefore, the frequency shift in each element is kept low, and the display color of each cell 71 is set to a display color of a low frequency shift in display image 70 of FIG. 9(a). Further, even if any cell region is designated in display image 70, a graph of FIG. 9(b) converges to a frequency of around zero.

On the other hand, in a case where a microorganism is resistant to the drug, the growth of the microorganism proceeds in sample 200. Therefore, similar to display image 70 of FIG. 9(a), the frequency shift becomes high in a predetermined element region. Further, when the tester designates the element region, a graph similar to a graph L1 of FIG. 9(b) is displayed.

In this way, the tester can properly evaluate whether the microorganism is sensitive or resistant to the drug to be evaluated. The growth of bacteria can be evaluated with time by array sensor 10. In this way, the sensitivity of a microorganism to a drug having an inhibitory action on the growth in a concentration-dependent or time-dependent manner can be precisely evaluated. As the representative drug having such an inhibitory action in a concentration-dependent manner, aminoglycosides, and new quinolones are known, and as the representative drug having such an inhibitory action in a time-dependent manner, penicillins, cephems, and carbapenems are known.

In this regard, analysis unit 40 initiatively executes each step in the test method of FIG. 8, but the tester may initiatively execute each step. For example, steps of steps S11 and S16 may be conducted by a switch operation by the tester, or steps of steps S13 and S14 may be initiatively conducted by the tester without being prompted from analysis unit 40, and the effect may be input to analysis unit 40 after the completion of these steps. Alternatively, a series of steps of steps S11 to S19 may be automated without performing a manual operation by the tester.

### Example 1

By using as a control a specimen containing bacillus Calmette-Guerin (BCG, Mycobacterium bovis) being a difficult-to-culture bacterium, the inventors conducted verification experiments of growth and drug sensitivity of BCG by the test method above.

In this experiment, a control sample was prepared by mixing 200 μL of a BCG suspension in which BCG was mixed with a liquid culture medium (MGIT PANTA) with 400 μL of an unused liquid culture medium (MGIT PANTA). Further, 196 μL of the prepared control sample was mixed with 4 μL of streptomycin (SM) at a concentration of 25 mg/mL to prepare an SM sample for drug sensitivity evaluation. Furthermore, 199.8 mL of the above-prepared control sample was mixed with 0.2 μL of rifampicin (RFP) at a concentration of 50 mg/mL to prepare an RFP sample for drug sensitivity evaluation.

200 μL of the above-prepared control sample, 200 μL of the above-prepared SM sample, and 200 μL of the above-prepared RFP sample are introduced into different containers 60, respectively, capping solvent 300 was further added into each container 60, and a frequency shift was measured for each sample. In this case, a perfluoro compound (PFC) in which oxygen was dissolved was used as capping solvent 300. Pure oxygen was blown into the PFC at 5 L/min for 1 minute to oxygenate the PFC, and immediately after that, the PFC was added into container 60, and container 60 was sealed.

During the measurement, container 60 was stored in an incubator, and further the temperature of the sample in container 60 was kept constant by a Peltier element. The temperature inside the incubator was set at 37° C., and the temperature of the Peltier element was set at 38° C. The humidity in the incubator was set at 100%.

Analysis processing was performed on an element of which the initial frequency was within the range of 64 to 65 GHz, among 1488 elements on array sensor 10. The frequency shift of each element was repeatedly measured and calculated every minute. The measurement was continuously performed until 12 hours elapsed from the start of the measurement.

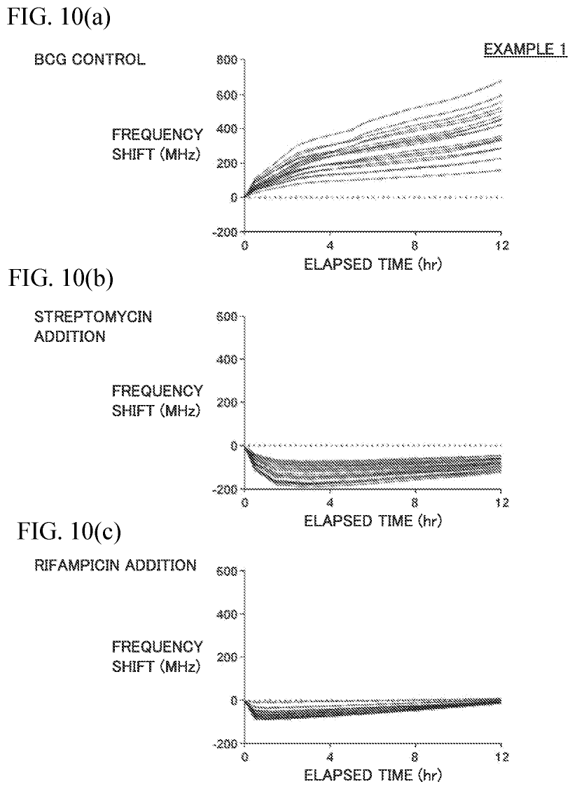

FIGS. 10(a) to 10(c) are graphs showing the experimental results of the drug sensitivity of BCG. FIG. 10(a) is a graph showing the experimental results for the control sample to which no drug was added, and FIGS. 10(b) and 10(c) are graphs showing the measurement results for the SM and RFP samples to which SM and RFP were added, respectively. These graphs can be displayed on monitor 41 in analysis unit 40.

FIG. 10(a) shows the measurement results in a case where 20 elements in which bacterial masses were confirmed by microscopic observation at a magnification of 500 times were used as the elements to be analyzed. FIGS. 10(b) and 10(c) show the measurement results in a case where 20 elements each having an initial frequency within the range of 64 to 65 GHz were used as the elements to be analyzed.

As shown in FIG. 10(a), as for the control sample to which no drug was added, in around half of the 20 elements set to be analyzed, the frequency shift steeply increased with the lapse of time, and converged to a frequency shift of around 300 to 600 MHz. Further, in the remaining around half of the 20 elements, the frequency shift gradually increased with the lapse of time, and converged to a frequency shift of around 100 to 200 MHz.

From these measurement results, it was able to be confirmed that a difference in the increase in the frequency shift occurred depending on the position of the element in the control sample to which no drug was added. In addition, this difference corresponded to the fact that the growth of BCG occurred locally in response to the masses of bacteria. According to this, it was able to be confirmed that the growth of BCG locally occurred in a sample was properly reflected on the frequency shift of each element.

Next, as shown in FIG. 10(b), as for the SM sample in which SM was added to a control sample, a steep increasing trend in the frequency shift with the lapse of time was not observed uniformly in any of the elements. From these results, it was able to be confirmed that in the SM sample, the growth of BCG was suppressed by a drug SM, and this was properly reflected on the frequency shift of each element.

Further, as shown in FIG. 10(c), as for the RFP sample in which RFP was added to a control sample, similar to the SM sample, a steep increasing trend in the frequency shift with the lapse of time was not observed uniformly in any of the elements. From these results, it was able to be confirmed that in the RFP sample, the growth of BCG was suppressed by a drug RFP, and this was properly reflected on the frequency shift of each element.

As described above, from the experimental results of Example 1, it was able to be verified that by the test method of the embodiment above, the growth of BCG and the drug sensitivities of BCG to SM and RFP were able to be confirmed based on the frequency shift of each element. Moreover, as shown in FIGS. 10(a) to 10(c), these confirmations were able to be made rapidly in only around 10 hours, and it was able to be confirmed that in particular, the time required for the drug sensitivity testing of BCG was able to be remarkably shortened as compared with the testing for drug sensitivity using a conventional agar medium or liquid culture medium. As described above, from the experimental results of Example 1, remarkable efficacy of the test method of the embodiment above was able to be confirmed.

### Example 2

By using NBRC3301 as a control strain of Escherichia coli, the inventors conducted verification experiments of growth and drug sensitivity of Escherichia coli by the test method above.

In this experiment, streptomycin (aminoglycoside) being a concentration-dependent drug was used. 196 μL of a control sample of NBRC3301 was mixed with 4 μL of SM at a concentration of 25 mg/mL to prepare a sample for drug sensitivity evaluation at a SM concentration of 500 μg/mL (SM_500 μg/mL). Further, the amount of the SM to be added to the control sample was adjusted to prepare a sample for drug sensitivity evaluation at a SM concentration of 250 μg/mL (SM_250 μg/mL) and a sample for drug sensitivity evaluation at a SM concentration of 125 μg/mL (SM_125 μg/mL). Furthermore, in order to verify the frequency shift of a killed bacterium, a sample was prepared by subjecting 200 μL of a control sample to heat treatment at 98° C. for 30 minutes. Moreover, in order to verify the frequency shift of a killed bacterium similarly, a phosphate buffered 4% formaldehyde solution was added to 200 μL of a control sample, and the obtained mixture was treated at room temperature for 10 minutes and then replaced with ultrapure water to prepare a sample (4% FA).

The samples thus prepared were introduced into different containers 60, respectively, and the frequency shift was measured for each sample. In this case, capping solvent 300 was not added into each container 60. The temperatures of an incubator and a Peltier element during the measurement were set in a similar manner to Example 1 above. In analysis processing, frequency shifts of all the 1488 elements on array sensor 10 were measured every 10 seconds, the average value of the measured frequency shifts was calculated every 5 minutes, and extracted as the frequency shift at each timing of the measurement. The measurement was continuously performed until 60 minutes elapsed from the start of the measurement.

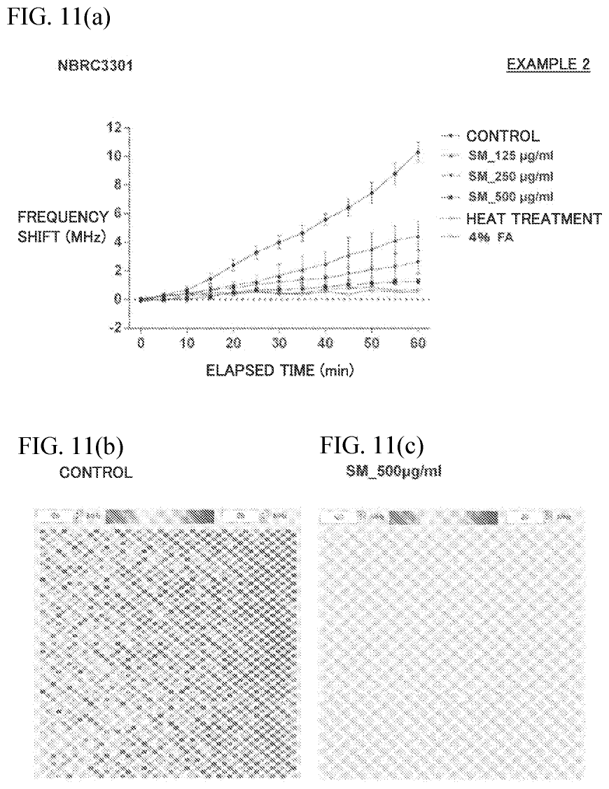

FIG. 11(a) is a graph showing the experimental results of the drug sensitivity of NBRC3301. The graph can be displayed on monitor 41 in analysis unit 40. Further, FIG. 11(b) is a display image showing a frequency shift of each element by color when a control sample to which SM was not added was measured at the time point after the lapse of 60 minutes, and FIG. 11(c) is a display image showing a frequency shift of each element by color when a sample for drug sensitivity evaluation at a SM concentration of 500 μg/mL (SM_500 μg/mL) was measured at the time point after the lapse of 60 minutes. In FIGS. 11(b) and 11(c), for convenience, the color images are shown in grayscale. In this case, the maximum frequency shift value of 20 MHz is set in blue, and the minimum frequency shift value of −20 MHz is set in red.

As shown in FIG. 11(a), as for the control sample to which SM was not added, the frequency shift greatly increased with the lapse of time. On the other hand, as for the three samples in each of which SM was added, the increase in the frequency shift was suppressed, and the higher the concentration of SM was, the greater the suppression of the increase in the frequency shift was, as compared with the control sample. Consequently, it was able to be confirmed that the drug sensitivity of SM to Escherichia coli was reflected on the frequency shift of each element in a drug concentration-dependent manner. This was also able to be confirmed by comparing the display images of FIGS. 11(b) and 11(c). That is, in the display image of a control sample shown in FIG. 11(b), elements with large frequency shifts were distributed substantially evenly, and in the display image of the SM_500 μg/mL sample shown in FIG. 11(c), the frequency shifts of all the elements were uniformly suppressed low.

Further, from the measurement results of FIG. 11(a), as for the killed bacteria that were subjected to heat treatment or formaldehyde treatment, it was able to be confirmed that the frequency shift almost did not occur in all the elements, and the fact that the growth of Escherichia coli did not occur was reflected on the frequency shift of each element. Therefore, it was able to be verified also from the measurement results that by the test method above, the growth of microorganisms was able to be confirmed by the frequency shift of each element.

As described above, from the experimental results of Example 2, it was able to be verified that by the test method of the embodiment above, the growth of Escherichia coli and the drug sensitivity of Escherichia coli to SM were able to be confirmed based on the frequency shift of each element.

### Example 3

By using NBRC12993 being a control strain of Staphylococcus epidermidis, the inventors conducted verification experiments of growth and drug sensitivity of Staphylococcus epidermidis by the test method above.

In this experiment, piperacillin (penicillin) being a time-dependent drug was used. That is, this drug exerts an inhibitory action on growth by retaining the effective drug concentration for a certain period of time. A control sample of NBRC12993 was mixed with piperacillin (PIPC) to prepare a sample for drug sensitivity evaluation at a PIPM concentration of 10.0 μg/mL (PIPC 10.0 μg/mL). Further, in order to verify the frequency shift of a killed bacterium, a sample was prepared by subjecting 200 μL of a control sample to heat treatment at 98° C. for 30 minutes.

The samples thus prepared were introduced into different containers 60, respectively, and the frequency shift was measured for each sample. In this case, capping solvent 300 was not added into each container 60. The temperatures of an incubator and a Peltier element during the measurement were set in a similar manner to Example 1 above. In analysis processing, frequency shifts of all the 1488 elements on array sensor 10 were measured every 10 seconds, the average value of the measured frequency shifts was calculated every 5 minutes, and extracted as the frequency shift at each timing of the measurement. In addition to this, in Example 3, a region where the growth of Staphylococcus epidermidis was observed was designated on the display image, and the average value of the frequency shifts in this growth region was calculated every 5 minutes. The measurement was continuously performed until 240 minutes elapsed from the start of the measurement.

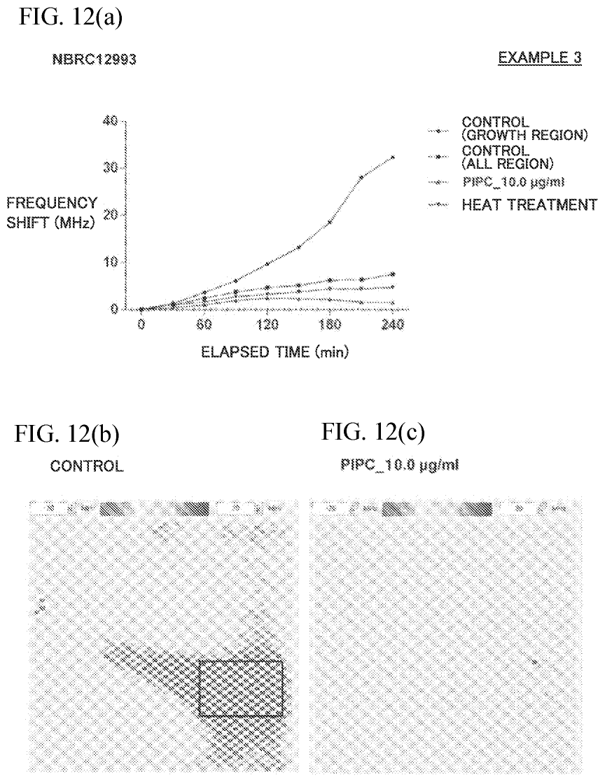

FIG. 12(a) is a graph showing the experimental results of the drug sensitivity of NBRC12993. In FIG. 12(a), the graph of “control (growth region)” shows the changes of the frequency shift calculated by designating the region where the growth of Staphylococcus epidermidis was observed on the display image, and the graph of “control (all region)” shows the changes of the frequency shift calculated by averaging the frequency shifts of all the elements. These graphs can be displayed on monitor 41 in analysis unit 40.

Further, FIG. 12(b) is a display image showing a frequency shift of each element by color when a control sample to which PIPC was not added was measured at the time point after the lapse of 240 minutes, and FIG. 12(c) is a display image showing a frequency shift of each element by color when a sample for drug sensitivity evaluation to which PIPC was added (PIPC 10.0 μg/mL) was measured at the time point after the lapse of 240 minutes. In FIGS. 12(b) and 12(c), for convenience, the color images are shown in grayscale. Similar to Example 2, in FIGS. 12(b) and 12(c), the frequency shift of greater than or equal to 20 MHz is set in blue, and the frequency shift of less than or equal to −20 MHz is set in red.

As shown in FIG. 12(a), as for the control sample to which PIPC was not added, in a case where the frequency shift was calculated by designating the growth region, the frequency shift greatly increased with the lapse of time. On the other hand, as for the sample to which PIPC was added, the increase in the frequency shift was suppressed more than that in the case where heat treatment was performed. Consequently, it was able to be confirmed that the drug sensitivity of PIPC to Staphylococcus epidermidis was reflected on the frequency shift of each element. This was also able to be confirmed by comparing the display images of FIGS. 12(b) and 12(c). That is, in the display image of a control sample shown in FIG. 12(b), elements with large frequency shifts were spread in a chain, and in the display image of the PIPC 10.0 μg/mL sample shown in FIG. 12(c), the frequency shifts of all the elements were uniformly suppressed low.

Further, from the measurement results of FIG. 12(a), as for the killed bacteria that were subjected to heat treatment, it was able to be confirmed that the frequency shift almost did not occur in all the elements, and the fact that the growth of Staphylococcus epidermidis did not occur was reflected on the frequency shift of each element. Therefore, it was able to be verified also from the measurement results that by the test method above, the growth of microorganisms was able to be confirmed by the frequency shift of each element.

Further, from the measurement results of FIG. 12(b), it was able to be confirmed that in a case of Staphylococcus epidermidis, the Staphylococcus epidermidis was spread in a chain and proliferated in a region in the vicinity of array sensor 10 even without using capping solvent 300. For this reason, as shown in FIG. 12(a), it was able to be confirmed that when the frequency shift was calculated by averaging the frequency shifts of all the elements, a difference from the case of killed bacteria (heat treatment) became small, and the growth of Staphylococcus epidermidis was not able to be accurately determined by the frequency shift. Therefore, it can be deemed that in a case where the test method of the embodiment above is applied to Staphylococcus epidermidis, it is necessary to designate the element region where growth is observed, and evaluate the growth of Staphylococcus epidermidis.

As described above, from the experimental results of Example 3, it was verified that by the test method of the embodiment above, the growth of Staphylococcus epidermidis and the drug sensitivity of Staphylococcus epidermidis to PIPC were able to be confirmed based on the frequency shift of each element.

Further, in FIG. 12(a), as for the sample to which PIPC was added, the frequency shift increased until around 120 minutes elapsed from the start of measurement, and the frequency shift showed a decreasing trend after the lapse of around 120 minutes. Consequently, the time-dependent action of PIPC on Staphylococcus epidermidis was able to be confirmed.

### Example 4

The inventors conducted an experiment of dissolving oxygen and nitrogen in a perfluoro compound (PFC) used as capping solvent 300 in Example 1 above.

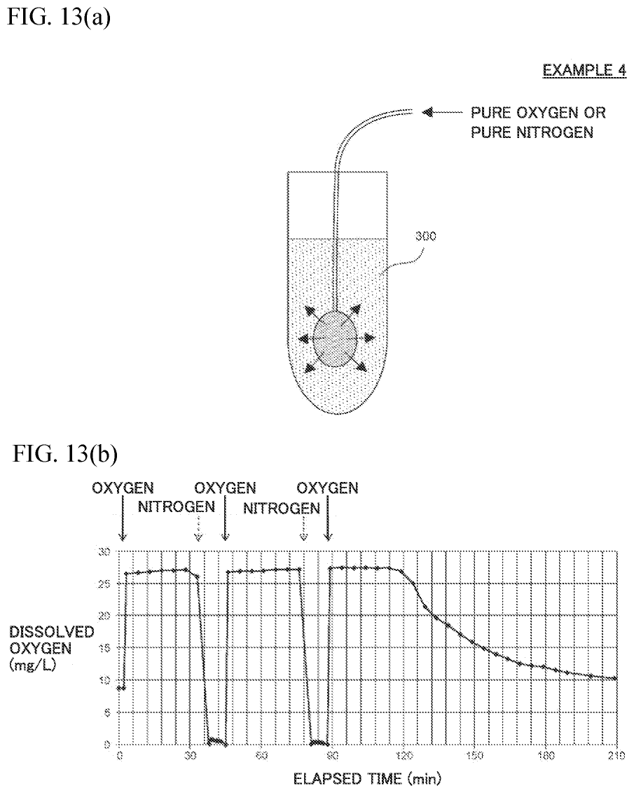

FIG. 13(a) is a diagram schematically showing the configuration used in an experiment of dissolution of oxygen or nitrogen in capping solvent 300. FIG. 13(b) is a graph showing the experimental results of dissolution of oxygen or nitrogen in capping solvent 300, according to Example 4.

As shown in FIG. 13(a), pure oxygen or pure nitrogen was blown into PFC being capping solvent 300 to be dissolved in the PFC. In this case, pure oxygen was blown into the PFC at a flow rate of 1 L/min for 2 minutes at the timings indicated by arrows with solid lines in FIG. 13(b), and pure nitrogen was blown into the PFC at a flow rate of 2 L/min for 5 minutes at the timings indicated by arrows with dashed lines in FIG. 13(b). The oxygen concentration in PFC was measured by using a predetermined measuring instrument.

As shown in FIG. 13(b), by blowing pure oxygen into the PFC, the concentration of pure oxygen in the PFC was able to be increased, and by blowing pure nitrogen into the PFC, the concentration of pure oxygen in the PFC was able to be decreased to around zero. Further, when pure oxygen was blown into the PFC, the oxygen concentration in the PFC was retained for around 30 minutes, and then the oxygen concentration in the PFC was gradually decreased. From these results, it was able to be confirmed that when PFC (capping solvent 300) was added in step S14 of FIG. 8 to enclose sample 200 with the PFC, oxygen in the PFC was eluted into the sample, and the oxygen was able to be supplied to the sample.

### Effect of Embodiment 1

As in the above, according to the present embodiment, by applying sample 200 to resonator 110 that oscillates in a gigahertz band and calculating the shift of the resonance frequency, the degree of growth of microorganisms in sample 200 can be rapidly and accurately tested.

At this time, as described with reference to FIG. 6(b), by setting the oscillation frequency of resonator 110 to greater than or equal to 10 GHz and less than or equal to 600 GHz, the influence of the ions existing in sample 200 on the shift of the resonance frequency of resonator 110 can be suppressed, and the shift range of the resonance frequency with the growth of microorganisms can be made larger.

Further, as described with reference to FIG. 6(b), by setting the oscillation frequency of resonator 110 to greater than or equal to 30 GHz and less than or equal to 300 GHz, the influence of the ions existing in sample 200 can be almost suppressed, and resonator 110 is easy to oscillate accurately at a predetermined resonance frequency.

Further, by applying sample 200 to array sensor 10 in which elements that include resonators 110 are arranged adjacently in a matrix pattern, the degree of growth of microorganisms can be tested for each position of the elements, as shown in Examples 1 and 3. In addition, as shown in FIGS. 11(b), 11(c), 12(b), and 12(c), distribution of the frequency shift of each element in the entire detection range of array sensor 10 can also be tested.

Further, by enclosing part of sample 200 in a region in the vicinity of resonator 110 with capping solvent 300, the influence of convection or the like due to temperature change is reduced, and sample 200 can be kept in the region in the vicinity of resonator 110. Therefore, as shown in Example 1 above, even if the microorganism to be tested is BCG or the like, the frequency shift corresponding to the degree of growth of microorganisms in sample 200 can be calculated accurately, and the testing of the microorganisms can be accurately conducted.

Further, in a case where the microorganisms in a sample are aerobic, by dissolving oxygen in capping solvent 300, and then adding capping solvent 300 to sample 200, the oxygen can be supplied to the microorganisms in the sample enclosed with capping solvent 300.

Further, as shown in Examples 1 to 3 above, in a case where the microorganism to be tested is Mycobacterium tuberculosis, Escherichia coli, or Staphylococcus epidermidis, the growth of the microorganism can be rapidly and accurately tested by the frequency shift of each element of array sensor 10.

### Embodiment 2

In Embodiment 1 above, in a case of enclosing sample 200 with capping solvent 300, sample 200 was introduced into container 60, and then capping solvent 300 was added to sample 200. On the other hand, in Embodiment 2, first, capping solvent 300 was introduced into container 60, and then an insertion tool was inserted into capping solvent 300, and after that, sample 200 was introduced through the insertion tool and enclosed in a region in the vicinity of array sensor 10.

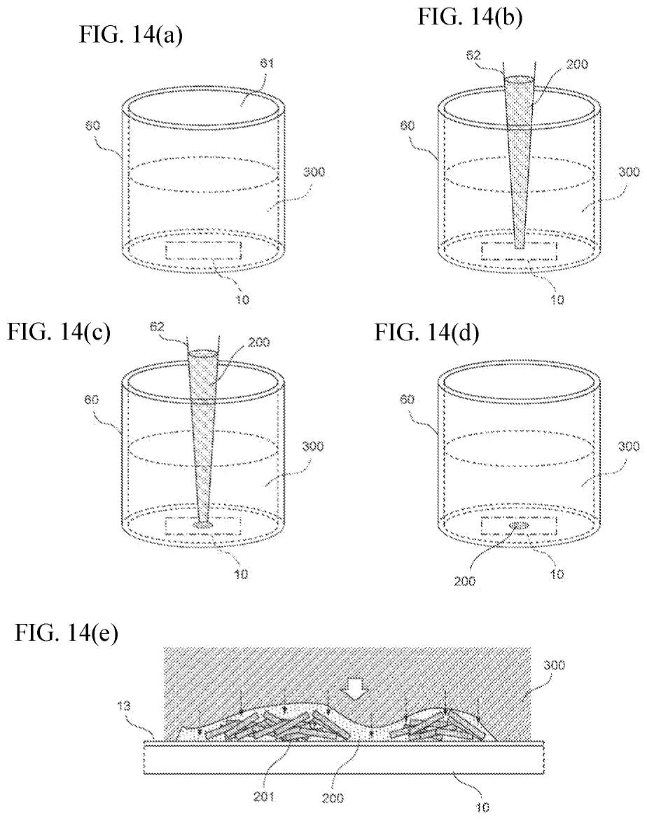

FIGS. 14(a) to 14(d) are diagrams showing steps of enclosing sample 200, respectively, according to Embodiment 2. FIG. 14(e) is a sectional view schematically showing a state of sample 200 enclosed with capping solvent 300.

First, as shown in FIG. 14(a), capping solvent 300 is introduced into container 60. Similar to Embodiment 1, array sensor 10 is arranged on a bottom surface of container 60 in a state that the top surface of array sensor 10 is covered with protective film 13 (see FIG. 4(a)).

Next, as shown in FIG. 14(b), an insertion tool 62 is inserted into capping solvent 300 stored in container 60. At this time, the exit of insertion tool 62 is positioned so that sample 200 can be introduced in a region in the vicinity near the surface of array sensor 10. In this state, sample 200 stored in insertion tool 62 is extruded from insertion tool 62 only by a predetermined amount. In this way, as shown in FIG. 14(c), sample 200 is added in the region in the vicinity near the surface of array sensor 10.

After that, extrusion of sample 200 is completed, and insertion tool 62 is withdrawn from capping solvent 300. Consequently, as shown in FIG. 14(d), sample 200 is positioned on the lower side of capping solvent 300 while being retained near the surface of array sensor 10. In this way, as shown in FIG. 14(e), sample 200 is enclosed in a region in the vicinity of above array sensor 10 with capping solvent 300, and microorganisms 201 in sample 200 are retained in the region in the vicinity of array sensor 10.

At this moment, by the pressure of capping solvent 300 due to own weight, microorganisms 201 together with sample 200 are pressed against the upper surface of array sensor 10 (protective film 13). Further, in a case where the microorganisms in sample 200 are aerobic, the gas (oxygen) dissolved in capping solvent 300 is eluted into sample 200, and the oxygen is supplied to microorganisms 201 in sample 200. In this way, the growth of microorganisms can be accurately tested by a frequency shift of each resonator 110 of array sensor 10.

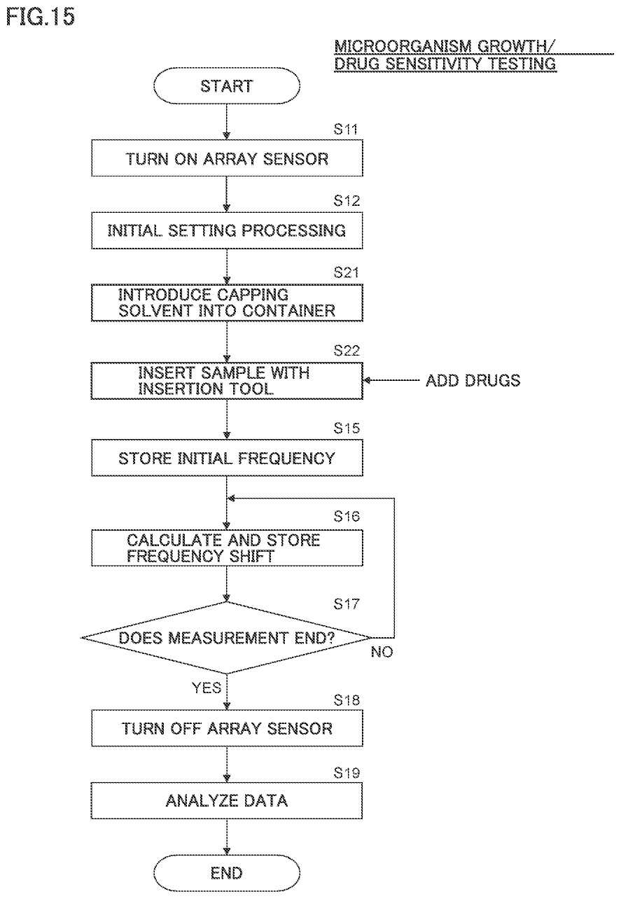

FIG. 15 is a flowchart showing steps of the microorganism test method.

In the flowchart of FIG. 15, steps S13 and S14 in the flowchart of FIG. 8 are replaced with steps S21 and S22, respectively. Processes in other steps of the flowchart of FIG. 15 is similar to the processes in corresponding steps of FIG. 8.

When the initial setting processing in step S12 is completed, analysis unit 40 prompts the tester to introduce capping solvent 300 into container 60 via a display screen. In response to this, the tester discards from container 60 the ultrapure water introduced into container 60 in the initial setting processing in step S12, and introduces capping solvent 300 into container 60 (S21).

In this way, when capping solvent 300 is introduced into container 60, analysis unit 40 further prompts the tester via a display screen or the like to add into container 60 sample 200 prepared by mixing a pretreated specimen with a liquid culture medium suitable for the microorganism to be tested. In response to this, as shown in FIGS. 14(b) and 14(c), the tester inserts insertion tool 62 such as a pipette into capping solvent 300, and introduces sample 200 to add sample 200 near the surface of array sensor 10 (S22). Consequently, as shown in FIG. 14(e), sample 200 is enclosed in a region in the vicinity of array sensor 10 with capping solvent 300.

After that, analysis unit 40 executes the processes after step S15 in a similar manner to Embodiment 1 above. In this way, analysis unit 40 measures the frequency shift in each element of array sensor 10 at predetermined time intervals, and stores the measurement results in a storage medium (S15 to S17). Further, when the measurement is completed (S17: YES), analysis unit 40 stops array sensor 10 (S18), and executes analysis processing of data accumulated during the measurement (S19). According to this analysis processing, for example, the images shown in FIGS. 9(a) and 9(b) are displayed on a display 41.

In this regard, in a case where the drug sensitivity of a microorganism is tested, a drug to be tested is mixed with sample 200 to be added in step S22.

In Embodiment 2, in a similar manner to Embodiment 1 above, the degree of growth of microorganisms in sample 200 can be rapidly and accurately tested.

Further, in Embodiment 2, as shown in FIGS. 14(a) to 14(d), capping solvent 300 is introduced into container 60, and then sample 200 is added through insertion tool 62 so that an adequate amount of sample 200 can be added with pinpoint accuracy in a region in the vicinity of array sensor 10. Accordingly, the amount of sample 200 to be added can be suppressed, and the enclosing region of sample 200 can be limited to a region in the vicinity of array sensor 10. Therefore, even in a case where a collected specimen is small in amount, the degree of growth of microorganisms in the specimen can be rapidly and accurately tested.

### Embodiment 3

In Embodiment 3, a configuration example in a case of automating the processes of FIGS. 8 and 15 is shown.

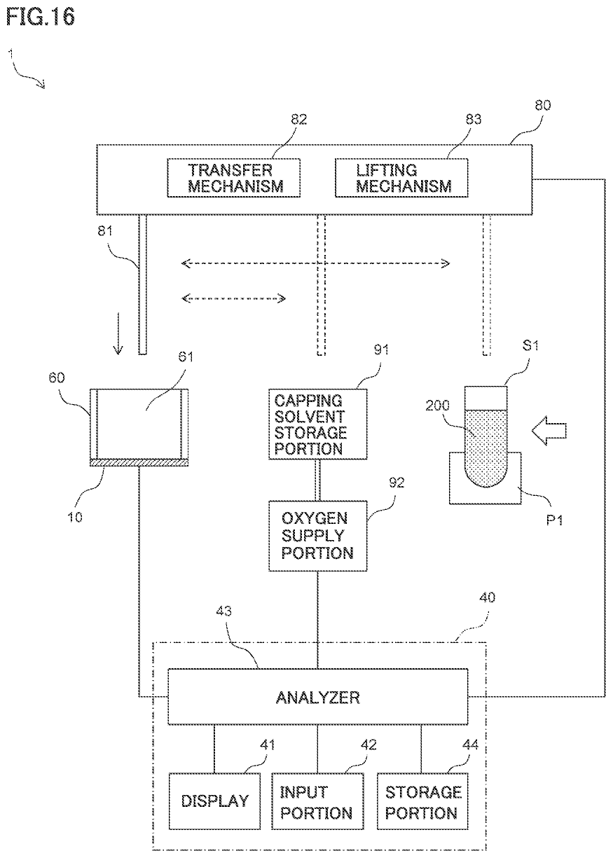

FIG. 16 is a block diagram showing the configuration of microorganism test apparatus 1 in a case of automating the processes of FIGS. 8 and 15.

As shown in FIG. 16, microorganism test apparatus 1 includes an introduction portion 80, a capping solvent storage portion 91, and an oxygen supply portion 92, in addition to array sensor 10, analysis unit 40, and container 60.

In a similar manner to Embodiment 1 above, array sensor 10 is attached to a bottom surface of container 60. The inside of container 60 is housing 61 for storing a liquid therein. For convenience, illustration of protective film 13 is omitted in FIG. 16.

Analysis unit 40 includes an input portion 42, an analyzer 43, and a storage portion 44, in addition to display 41. Input portion 42 includes input means such as operation buttons, a keyboard, and a mouse. Input portion 42 may be a touch panel.

Analyzer 43 has an arithmetic processing circuit such as a central processing unit (CPU), and controls each portion in accordance with a program stored in storage portion 44. Storage portion 44 includes storage media such as read only memory (ROM), random access memory (RAM), and a hard disk, and stores a program for control by analyzer 43, and data of the frequency shift described above and the like. Further, storage portion 44 is also used as a work area for processing of analyzer 43.

Introduction portion 80 includes a nozzle 81 for sucking and discharging a liquid. Further, introduction portion 80 includes a transfer mechanism 82 for transferring nozzle 81 to the installation position of a sample container S1 storing sample 200, the arrangement position of capping solvent storage portion 91 retaining capping solvent 300, and the arrangement position of container 60, and a lifting mechanism 83 for moving up and down nozzle 81 at each position.

Capping solvent storage portion 91 retains a predetermined amount of capping solvent 300, which is larger than the amount to be introduced into housing 61. Capping solvent storage portion 91 is connected to a tank (not shown) for storing a large amount of capping solvent 300, and is supplied with capping solvent 300 from the tank so as to be filled in the predetermined amount.

Oxygen supply portion 92 is connected to capping solvent storage portion 91 with a pipe, and supplies oxygen to capping solvent 300 retained in capping solvent storage portion 91. As described above, when oxygen is supplied to capping solvent 300, the oxygen dissolves in capping solvent 300, and the oxygen concentration of capping solvent 300 is increased. Oxygen supply portion 92 is connected to an oxygen cylinder (not shown).

At the time of measurement, the tester inputs an instruction to start the measurement via input portion 42. At this time, at the same time, the tester inputs whether or not to use capping solvent 300. Alternatively, the tester may input the kind of microorganism to be tested so as to allow analyzer 43 to determine whether or not to use capping solvent 300. In this case, for example, information that associates the kind of microorganism with the necessity of using capping solvent 300 is stored in advance in storage portion 44. Analyzer 43 determines whether or not to use capping solvent 300 based on the information.

In response to the above input, analyzer 43 executes the processes in steps S11 and S12 of FIG. 8 or 15. After that, analyzer 43 outputs on display 41 a display that prompts the tester to install sample container S1 storing sample 200 in an installation portion P1 in the apparatus. In response to this, the tester installs sample container S1 in installation portion P1. The installation of sample container S1 is detected, for example, by a sensor arranged in installation portion P1. Alternatively, the input indicating that sample container S1 is installed may be performed via input portion 42 by the tester.

In this way, when sample container S1 is installed, analyzer 43 controls introduction portion 80 to introduce sample 200 and capping solvent 300 into housing 61 of container 60. In a case where measurement is performed without using capping solvent 300, analyzer 43 controls introduction portion 80 so that only sample 200 is introduced into container 60.

In a case where measurement is performed in accordance with the flowchart of FIG. 8, analyzer 43 first lets nozzle 81 down into sample container S1 to suck a predetermined amount of sample 200, and then discharge the sucked sample 200 into housing 61 of container 60 (S13). Next, analyzer 43 lets nozzle 81 down into capping solvent storage portion 91 to suck a predetermined amount of capping solvent 300, and then discharge the sucked capping solvent 300 into housing 61 of container 60 (S14). As a result, as shown in FIGS. 7(a) to 7(e), capping solvent 300 sinks, and sample 200 is enclosed in a region in the vicinity of array sensor 10 with capping solvent 300.

In a case where measurement is performed in accordance with the flowchart of FIG. 15, first, analyzer 43 lets nozzle 81 down into capping solvent storage portion 91 to suck a predetermined amount of capping solvent 300, and then discharge the sucked capping solvent 300 into housing 61 of container 60 (S21). Next, analyzer 43 lets nozzle 81 down into sample container S1 to suck a predetermined amount of sample 200, and then discharge the sucked sample 200 to a position close to array sensor 10 in container 60 (S22).

In this case, sample 200 is inserted by using nozzle 81 as insertion tool 62 of FIG. 14(b). Specifically, by letting nozzle 81 down into housing 61 of container 60, nozzle 81 is insert into capping solvent 300. At this time, nozzle 81 is let down until the tip of nozzle 81 reaches a position close to the surface of array sensor 10. In this state, sample 200 sucked by nozzle 81 is discharged from nozzle 81, and then nozzle 81 is withdrawn from housing 61. As a result, as shown in FIGS. 14(a) to 14(e), sample 200 is enclosed in a region in the vicinity of array sensor 10 with capping solvent 300.

After that, analyzer 43 executes the processes in steps S15 to S17 of FIG. 8 or 15, calculates the frequency shift of each element at predetermined time intervals, and stores the calculated frequency shift in storage portion 44. Further, when the measurement is completed (S17: YES), analyzer 43 stops the operation of array sensor 10 (S18), and analyzes the degree of growth of microorganisms in a specimen based on the frequency shift stored in storage portion 44. The analysis method is similar to that in Embodiment 1 above.

According to the configuration of Embodiment 3, the processes of FIGS. 8 and 15 are automated, and thus the testing of a microorganism can be more easily conducted.

In this regard, in the configuration of FIG. 16, sample container S1 storing sample 200 is installed in installation portion P1 by the tester, but sample container S1 may be transported to a suction position of sample 200 by a transport means.

Further, in the configuration of FIG. 16, sample container S1 that stores sample 200 prepared outside is installed in microorganism test apparatus 1, but a sample preparation portion that prepares a sample by mixing a specimen with a liquid culture medium may further be arranged in microorganism test apparatus 1. In this case, a specimen container storing a specimen is installed in installation portion P1, and the specimen is sucked from the specimen container by nozzle 81. The sucked specimen is discharged to the sample preparation portion, and mixed with a reagent and a liquid culture medium. In this way, a sample for measurement is prepared in the sample preparation portion. After that, the sample is sucked from the sample preparation portion by a nozzle and discharged into container 60.

Further, in the configuration of FIG. 16, only one set of container 60 and array sensor 10 is arranged, but multiple sets of container 60 and array sensor 10 may be arranged in microorganism test apparatus 1. In this case, for example, by introducing multiple samples 200 to which different drugs were added, into different containers 60, respectively, the degree of growth of microorganisms can be measured in parallel for each drug. In this way, the drug sensitivity of a microorganism for each drug can be more rapidly confirmed.

Further, analysis unit 40 may not be configured as a separate personal computer as in the configuration of FIG. 1, or may be integrally equipped with microorganism test apparatus 1. In this case, display 41, input portion 42, analyzer 43, and storage portion 44 may not be unitized, and may be individually arranged in microorganism test apparatus 1.

### Embodiment 4

In Embodiment 1 above, a fluorine-based inert solvent such as a perfluoro compound (PFC) was used as hydrophobic capping solvent 300. On the other hand, mineral oil was used in Embodiment 4 as hydrophobic capping solvent 300. Mineral oil is also referred to as liquid paraffin. Mineral oil having a specific gravity lower than that of water is used as capping solvent 300. The specific gravity of the mineral oil to be used is, for example, around 0.85. Mineral oil having a specific gravity higher than that of water may be used.

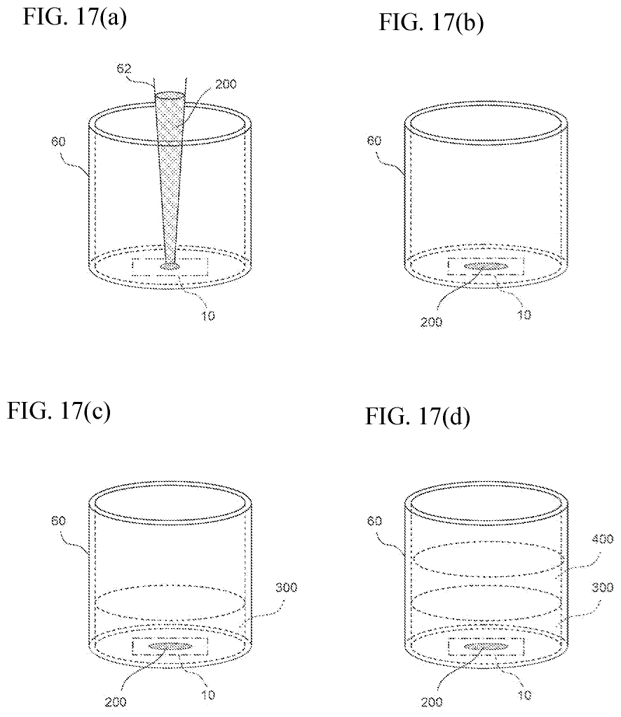

FIGS. 17(a) to 17(d) are diagrams showing steps of adding capping solvent 300 to sample 200, respectively.

First, as shown in FIGS. 17(a) and 17(b), sample 200 is added in a region in the vicinity near the surface of array sensor 10. Next, as shown in FIG. 17(c), capping solvent 300 made of mineral oil is introduced into container 60. Capping solvent 300 encloses sample 200 so as to cover sample 200 without being mixed with sample 200 because of being hydrophobic. After that, as shown in FIG. 17(d), a water 400 is introduced into container 60 so as to overlap the upper surface of capping solvent 300. In this case, because of the hydrophobicity of capping solvent 300, water 400 deposits on the upper surface of capping solvent 300 without being mixed with capping solvent 300.

The step of adding capping solvent 300 in a case where the mineral oil is capping solvent 300 is not limited to the above, and other methods may be used as long as sample 200 can be enclosed with capping solvent 300. Further, also in a case where mineral oil is used as capping solvent 300, the addition of capping solvent 300 to sample 200, and the measurement and analysis of the sample can be automatically performed by an apparatus similar to that of FIG. 16.

### Example 5

The inventors measured the degree of growth of BCG (Mycobacterium bovis) by using mineral oil as capping solvent 300. Sample 200 to be used for measurement was prepared as follows.

After stirring a BCG suspension prepared by mixing BCG with a liquid culture medium (MGIT PANTA) with a mixer, the stirred BCG suspension was left to stand at ordinary temperature for 15 minutes, the obtained supernatant was diluted 10 time with a new liquid culture medium, and the diluted supernatant was stirred again with a mixer. After that, the BCG suspension was left to stand at ordinary temperature for 15 minutes, the supernatant was collected and stirred again with a mixer, and the stirred BCG suspension was adjusted to 1000 CFU/μL by a cell counter for bacteria. In this way, sample 200 to be used for measurement was prepared.

Sample 200 thus prepared was enclosed in a region in the vicinity near the surface of array sensor 10 with capping solvent 300 made of mineral oil, by the method shown in FIGS. 17(a) to 17(d). After that, while culturing BCG in sample 200, the output from array sensor 10 was analyzed, and the degree of growth of the BCG was calculated in a similar manner to Example 1 above (Example 5).

Further, as for the case where a perfluoro compound (PFC) similar to that in Example 1 above was used as capping solvent 300, similar measurement and analysis were performed (Example 6). In the measurement and analysis, sample 200 was enclosed with capping solvent 300 in a similar manner to that in FIGS. 17(a) to 17(d).

In addition, as for the case where capping solvent 300 was not used, similar measurement and analysis were performed (Example 7). In the measurement and analysis, sample 200 was only introduced into container 60.

In each measurement (Examples 5 and 6) using capping solvent 300 (mineral oil/PFC), 5 μL of sample 200 was introduced into container 60. Further, in the measurement using no capping solvent (Example 7), 300 μL of sample 200 was introduced into container 60.

In a case where a perfluoro compound (PFC) was used as capping solvent 300 (Example 6), oxygen was dissolved in capping solvent 300 in a similar manner to Example 1 above. That is, pure oxygen was blown into PFC at 5 L/min for 1 minute to oxygenate the PFC, and immediately after that, the PFC was added into container 60, and container 60 was sealed. In a case where mineral oil was used as capping solvent 300 (Example 5), oxygen was not dissolved in capping solvent 300.

In each measurement, in a similar manner to Example 1 above, 20 elements in which bacterial masses were confirmed by microscopic observation at a magnification of 500 times were used as the elements to be analyzed. In this regard, in the measurement using PFC as capping solvent 300 (Example 6), further, 20 elements on each of which capping solvent 300 positioned outside the region in the vicinity was placed were added to the elements to be analyzed. In each measurement, the frequency shifts of these elements to be analyzed were measured and calculated repeatedly every 10 minutes. Each measurement was continuously performed until 168 hours (7 days) elapsed from the start of the measurement.

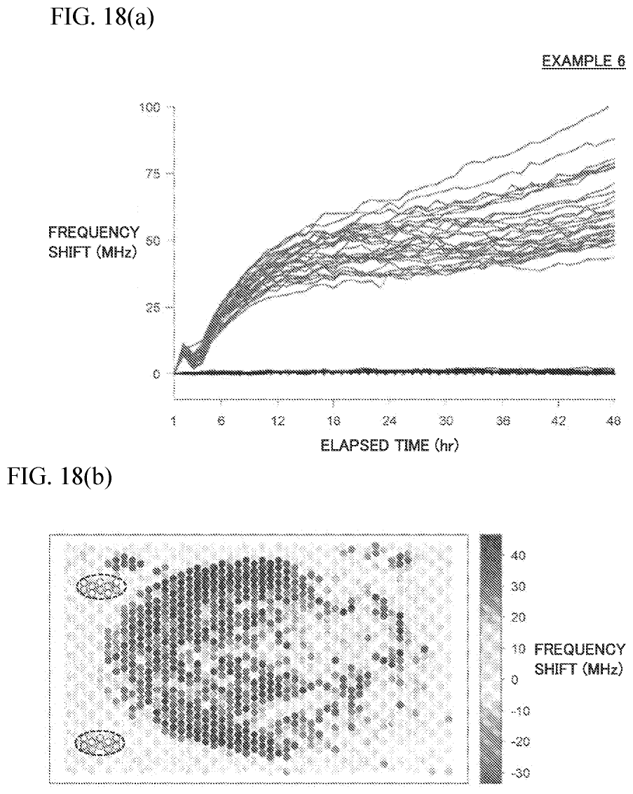

FIG. 18(a) is a graph showing frequency shifts of the elements to be analyzed of Example 6 in which PFC was used as capping solvent 300. In this case, the measurement results until 48 hours elapsed from the start of the measurement are shown. FIG. 18(b) is a display image showing a frequency shift of each element by color at the time point after the lapse of 168 hours (7 days) in Example 6 in which PFC was used as capping solvent 300.

In FIG. 18(b), for convenience, the color images are shown in grayscale. In this case, the maximum frequency shift value (herein 40 MHz) is set in blue, and the minimum frequency shift value (herein −30 MHz) is set in red. In FIG. 18(b), 20 elements surrounded with a dashed line are 20 elements on each of which capping solvent 300 positioned outside the above-described region in the vicinity was placed. The calculation results of frequency shifts of these elements are shown in FIG. 18(a) as a graph that shifts finely around zero.

As shown in FIG. 18(a), in a case where PFC was used as capping solvent 300, the frequency shifts in 20 elements (in which bacterial masses were confirmed) contained in a region enclosed with capping solvent 300 were increased with the lapse of time similar to Example 1 above. Therefore, also from these measurement results, it was able to be confirmed that the growth of BCG locally occurred in sample 200 was properly reflected on the frequency shift of each element.

Further, with reference to the measurement results of FIG. 18(b), a region where sample 200 was enclosed with capping solvent 300 was greatly spread out in an elliptical shape in the middle of the image, and elements with large frequency shifts were distributed in this region. Further, outside this region, there were only a few scattered elements with large frequency shifts, and in most of the elements, there was substantially no frequency shift or was only slight frequency shift. From these results, it was able to be confirmed that by enclosing sample 200 with the use of PFC as capping solvent 300, the degree of growth of BCG was able to be efficiently and accurately detected from the output of the elements in the enclosing region.

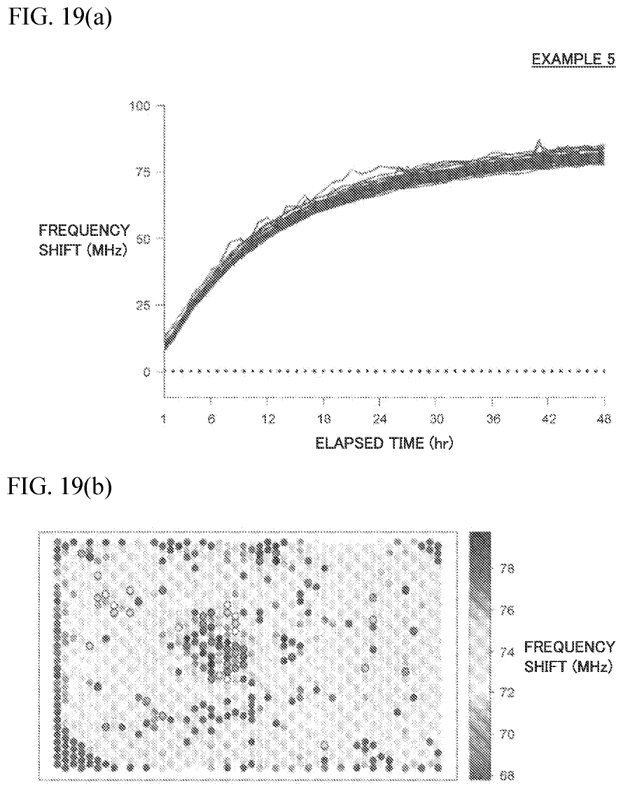

FIG. 19(a) is a graph showing frequency shifts of the elements to be analyzed of Example 5 in which mineral oil was used as capping solvent 300. In this case, the measurement results until 48 hours elapsed from the start of the measurement are shown. FIG. 19(b) is a display image showing a frequency shift of each element by color at the time point after the lapse of 168 hours (7 days) in Example 5 in which mineral oil was used as capping solvent 300.

Similar to FIG. 18(b), also in FIG. 19(b), for convenience, the color images are shown in grayscale.

Further, in a case where mineral oil was used as capping solvent 300, as compared with the case where PFC was used as capping solvent 300, the area of the enclosing region where sample 200 was enclosed is increased, and the width (thickness) in the height direction of the enclosing region is decreased. Therefore, in the image of FIG. 19(b), the enclosing region of sample 200 is spread over the range of substantially all of the elements except for part of the range at the lower left corner and in the vicinity of the left side, and the boundary of an elliptical shape indicating the enclosing region as shown in FIG. 18(b) is not observed on the image.

As shown in FIG. 19(a), in a case where mineral oil was used as capping solvent 300, the frequency shifts in 20 elements (in which bacterial masses were confirmed) contained in a region enclosed with capping solvent 300 were increased with the lapse of time similar to Example 1 above. Therefore, also in a case where mineral oil was used as capping solvent 300, it was able to be confirmed that the growth of BCG locally occurred in sample 200 was properly reflected on the frequency shift of each element.

Further, from the measurement results of FIG. 19(b), a region where elements with large frequency shifts were densely distributed was confirmed in a region where sample 200 was enclosed with capping solvent 300. From this, it was able to be confirmed that by enclosing sample 200 with the use of mineral oil as capping solvent 300, the degree of growth of BCG was able to be accurately detected.

In addition, in the measurement results of FIG. 19(a), it was able to be confirmed that the changes of the frequency shift with the lapse of time were less likely to vary between elements as compared with the measurement results of FIG. 18(a). As described above, this can be considered to be because in a case where mineral oil was used as capping solvent 300, the width (thickness) in the height direction of the enclosing region of sample 200 by capping solvent 300 is smaller as compared with the case where PFC was used as capping solvent 300 so that BCG is less likely to flow away from the element in the enclosing region. Therefore, it can be considered that by using mineral oil as capping solvent 300, the measurement results of more stable frequency shifts can be obtained from the elements to be measured.

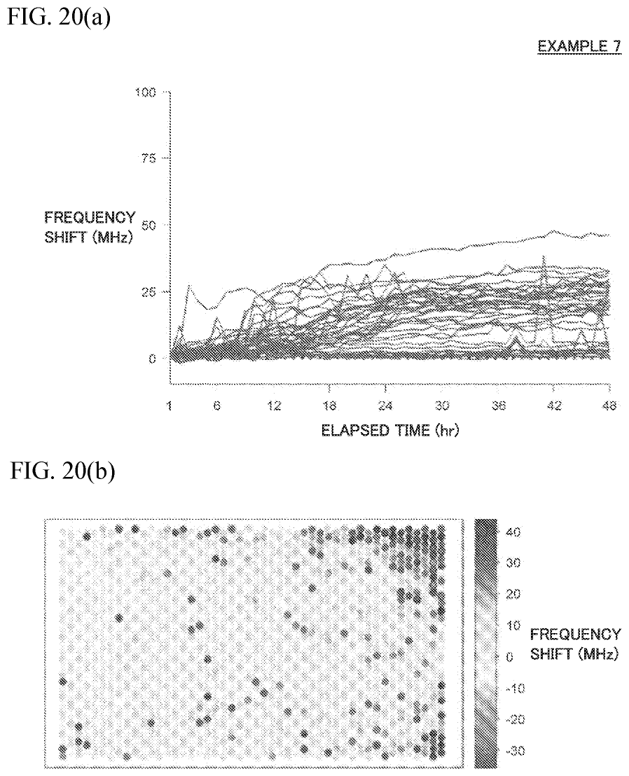

FIG. 20(a) is a graph showing frequency shifts of the elements to be analyzed of Example 7 in which capping solvent 300 was not used. In this case, the measurement results until 48 hours elapsed from the start of the measurement are shown. FIG. 20(b) is a display image showing a frequency shift of each element by color at the time point after the lapse of 168 hours (7 days) in Example 7 in which capping solvent 300 was not used.

As shown in FIG. 20(a), in a case where capping solvent 300 was not used, the frequency shift in each element was not able to be stably detected. Therefore, it was able to be confirmed that in a case where an object to be analyzed was BCG, it was difficult to reflect the degree of growth of BCG on the frequency shift of each element without using capping solvent 300.

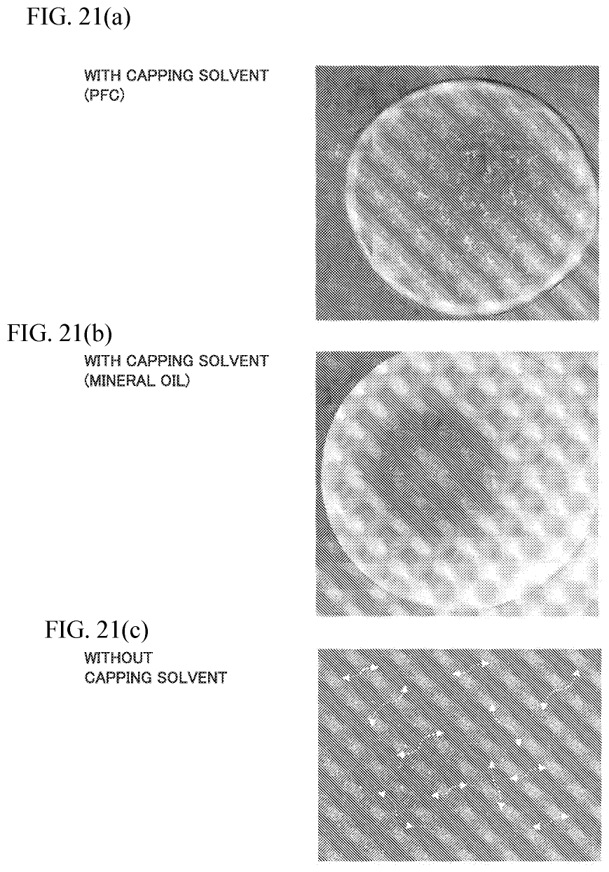

FIGS. 21(a) to 21(c) are photomicrographs showing the case where PFC was used as capping solvent 300 (Example 6), the case where mineral oil was used as capping solvent 300 (Example 5), and the case where capping solvent 300 was not used (Example 7), respectively. In FIGS. 21(a) and 21(b), regions where sample 200 was enclosed with capping solvent 300 are observed. The photomicrograph of FIG. 21(b) was taken by a microscope with a higher magnification as compared with those of the other photomicrographs.

In the microscopic observation of FIG. 21(c), as schematically indicated by arrows with dashed lines, because bacterial masses flow in a liquid medium, the growth of bacteria as a whole can be evaluated, but the growth of individual bacterial cells cannot be evaluated. On the other hand, in the microscopic observations shown in FIGS. 21(a) and 21(b), because capping solvent 300 is used, the flow of bacterial masses is less. For this reason, the microscopic observation of bacterial masses can be performed precisely over a long period of time. Therefore, by using capping solvent 300, division and growth of individual bacterial cells can be observed with time.

Further, as shown in FIG. 21(b), in a case where mineral oil was used as capping solvent 300, the resolution of the photomicrograph is slightly lowered in relation to the refractive index of mineral oil. On the other hand, as shown in FIG. 21(a), in a case where PFC was used as capping solvent 300, the high resolution of the photomicrograph is maintained. Therefore, it can be deemed that it is preferable to use PFC as capping solvent 300 in a case where growth of bacterial cells is observed under a microscope.

In addition to this, in a case where capping solvent 300 is used, the drying of sample 200 can be prevented because sample 200 is enclosed with capping solvent 300. Further, in a case where capping solvent 300 is used, growth of microorganisms in sample 200 can be maintained because gas such as oxygen is exchanged between sample 200 and capping solvent 300. From these things, in a case where capping solvent 300 is used, the degree of growth of microorganisms can be stably measured over a long period of time.

### Change Example

Although the embodiments of the present invention have been specifically described above, the present invention is not limited to the embodiments described above, and the embodiments of the present invention can be modified in various ways other than those described above.

For example, the microorganism to be tested is not limited to the microorganisms shown in Examples 1 to 3 above, and may be other microorganisms. For example, an acid-fast bacterium such as M. africanum, M. gordonae, M. avium, M. intracellulare, M. kansasi, M. marinum, M. abscessus, M. microti, or M. ulcerance may be an object to be tested. The above test method can also be used for evaluating the drug sensitivity to unknown bacteria.

Further, a drug for evaluating the drug sensitivity of a microorganism is also not limited to the drugs shown in Examples 1 to 3 above, and the drug sensitivity of a microorganism to other drugs may be evaluated.

In addition, the test methods of the above embodiments may be used not only for the drug sensitivity of a microorganism but also for the evaluation of growth of microorganisms in food management. The present invention can be effectively used in the field of evaluating the growth of microorganisms.

Further, in the test methods of FIGS. 8 and 15, after the frequency shift of each element is accumulated until the measurement is completed, the accumulated frequency shifts are analyzed, and the analysis results regarding the growth of microorganisms are displayed on monitor 41, but the frequency shift may be analyzed in real time during the measurement, and the analysis results as shown in FIGS. 9(a) and 9(b) may be displayed on monitor 41 at any time.

In addition, the resonance frequency of each resonator 110 is stored in a storage portion of analysis unit 40 in time series of from the start of measurement to the end of measurement, and during the analysis, in analysis unit 40, the frequency shift between the resonance frequency at each timing stored in the storage portion and the resonance frequency at the time of starting the measurement may be calculated.

Further, in the above embodiment, sample 200 was applied to array sensor 10 (resonator 110) by introducing sample 200 into container 60 in which array sensor 10 was installed on the bottom part, but the method for applying sample 200 to array sensor 10 is not limited thereto. For example, sample 200 may be applied to array sensor 10 by immersing array sensor 10 in sample 200 stored in container 60.

In addition, the enclosing methods shown in FIGS. 7(a) to 7(e), and 14(a) to 14(e) are effective also in a case where a sensor other than the sensor that detects the frequency shift of resonator 110 is used. For example, another type of sensor IC made of a semiconductor may be used. Even in a case where another type of sensor is used, a sample can be enclosed in a region in the vicinity of the sensor by using the enclosing methods shown in FIGS. 7(a) to 7(e), 14(a) to 14(e), and 17(a) to 17(d), and the state of microorganisms in the sample can be accurately tested.

Further, the configuration of microorganism test apparatus 1 is not limited to the configurations shown in FIGS. 1 and 16, and any other configuration may also be accepted as long as it can enclose sample 200 with capping solvent 300 on array sensor 10. In addition, in microorganism test apparatus 1 for testing a microorganism that does not require the use of capping solvent 300, capping solvent storage portion 91 and oxygen supply portion 92 in FIG. 16 may be omitted.

In addition to this, the embodiments of the present invention can be modified in various ways.

### REFERENCE SIGNS LIST

1: microorganism test apparatus, 10: array sensor, 40: analysis unit (analyzer), 41: display, 43: analyzer, 60: container, 61: housing, 80: introduction portion, 110: resonator, 150: oscillator, 200: sample, 300: capping solvent, 201: microorganism

1. A microorganism test method, comprising:
covering, with a hydrophobic capping solvent, a sample containing a specimen and a liquid culture medium, within a region in a vicinity of a sensor, the sensor configured to detect a microorganism contained in the specimen; and
calculating, based on an output from the sensor, information indicating a degree of growth of the microorganism contained in the specimen.

2. The microorganism test method according to claim 1, wherein
the capping solvent is introduced in a storing region including the region in the vicinity of the sensor, an insertion tool is inserted into the capping solvent, the sample is introduced in the region in the vicinity of the sensor through the insertion tool to enclose the sample in the region in the vicinity of the sensor.

3. The microorganism test method according to claim 1, wherein
the sensor is an array sensor in which a plurality of sensor elements are arranged adjacently in a matrix pattern.

4. The microorganism test method according to claim 1, wherein
each of the sensor elements includes an oscillator that oscillates in a gigahertz band, and
shift of an oscillation frequency of the oscillator is calculated as the information indicating the degree of growth of the microorganism contained in the specimen.

5. The microorganism test method according to claim 1, wherein
a growth curve for evaluating shift with time in the growth of the microorganism is displayed in a display based on the output from the sensor.

6. The microorganism test method according to claim 1, wherein
the capping solvent has gas solubility, and
the sample is enclosed in the region in the vicinity of the sensor with the capping solvent in which oxygen is dissolved.

7. The microorganism test method according to claim 1, wherein
the capping solvent is a fluorine-based inert solvent, or mineral oil.

8. The microorganism test method according to claim 1, wherein
the microorganism includes Mycobacterium tuberculosis.

9. A microorganism test method, comprising:
applying a sample prepared by mixing a specimen with a liquid culture medium to a resonator that oscillates in a gigahertz band; and
calculating shift of a resonance frequency of the resonator as information indicating a degree of growth of a microorganism contained in the specimen.

10. The microorganism test method according to claim 9, wherein
the gigahertz band is greater than or equal to 10 GHz and less than or equal to 600 GHz.

11. The microorganism test method according to claim 10, wherein
the gigahertz band is greater than or equal to 30 GHz and less than or equal to 300 GHz.

12. The microorganism test method according to claim 9, wherein
the sample is applied to an array sensor in which elements that include the resonators are arranged adjacently in a matrix pattern.

13. The microorganism test method according to claim 9, wherein
the sample is enclosed in a region in a vicinity of the resonator with a capping solvent that hydrophobic.

14. The microorganism test method according to claim 13, wherein
the capping solvent has gas solubility, and
oxygen is dissolved in the capping solvent.

15. The microorganism test method according to claim 13, wherein
the capping solvent is a fluorine-based inert solvent, or mineral oil.

16. The microorganism test method according to claim 9, wherein
the microorganism to be tested includes at least one of Mycobacterium tuberculosis, Escherichia coli, and Staphylococcus epidermidis.

17. The microorganism test method according to claim 9, wherein
for the sample containing a drug for evaluating drug sensitivity to the microorganism, the resonance frequency of the resonator is calculated, and
a growth curve indicating shift with time of the calculated resonance frequency is displayed in a display.

18. The microorganism test method according to claim 9, wherein
a plurality of the samples that contain drugs at different concentrations from each other for evaluating the drug sensitivity to the microorganism are prepared,
the shift of the resonance frequency of the resonator is calculated for each sample, and
the calculated shift with time of the resonance frequency of each sample is displayed in the display.

19. A microorganism test apparatus, comprising:
a housing that stores a liquid;
an introduction portion that introduces, into the housing, a sample prepared by mixing a specimen with a liquid culture medium, and a capping solvent that is hydrophobic;
a sensor arranged close to the housing; and
an analyzer that calculates, based on an output from the sensor, information indicating a degree of growth of a microorganism contained in the specimen, wherein
the analyzer causes the introduction portion to enclose the sample in a region in a vicinity of the sensor with the capping solvent, and calculates information indicating the degree of growth.

20. A microorganism test apparatus, comprising:
a housing that stores a sample prepared by mixing a specimen with a liquid culture medium;
a resonator that is arranged close to the housing, and oscillates in a gigahertz band; and
an analyzer that calculates, based on a resonance frequency of the resonator, information indicating a degree of growth of a microorganism contained in the specimen.
